SEQUENCE LISTING (139 sequences)

SEQ ID NO: 1 - DNA, 23 bp, Artificial Sequence
  Synthetic
cctacacgacgctcttccgatct

SEQ ID NO: 2 - DNA, 25 bp, Artificial Sequence
  Synthetic
  misc_feature (7)..(7): r is a or g
cttaccrgagccattaaccaaatac

SEQ ID NO: 3 - DNA, 48 bp, Artificial Sequence
  Synthetic
  misc_feature (30)..(30): r is a or g
cctacacgacgctcttccgatctcttaccrgagccattaaccaaatac

SEQ ID NO: 4 - DNA, 25 bp, Artificial Sequence
  Synthetic
atctgaaggaagtgaacatcagcat

SEQ ID NO: 5 - DNA, 23 bp, Artificial Sequence
  Synthetic
gtgactggagttcagacgtgtgc

SEQ ID NO: 6 - DNA, 48 bp, Artificial Sequence
  Synthetic
atctgaaggaagtgaacatcagcatgtgactggagttcagacgtgtgc

SEQ ID NO: 7 - DNA, 39 bp, Artificial Sequence
  Synthetic
aatgatacggcgaccaccgagatctacacacactctttc

SEQ ID NO: 8 - DNA, 23 bp, Artificial Sequence
  Synthetic
cctacacgacgctcttccgatct

SEQ ID NO: 9 - DNA, 62 bp, Artificial Sequence
  Synthetic
aatgatacggcgaccaccgagatctacacacactctttccctacacgacgctcttccgatct

SEQ ID NO: 10 - DNA, 34 bp, Artificial Sequence
  Synthetic
caagcagaagacggcatacgagatgatcggaaga

SEQ ID NO: 11 - DNA, 23 bp, Artificial Sequence
  Synthetic
gcacacgtctgaactccagtcac

SEQ ID NO: 12 - DNA, 57 bp, Artificial Sequence
  Synthetic
caagcagaagacggcatacgagatgatcggaagagcacacgtctgaactccagtcac

SEQ ID NO: 13 - DNA, 13 bp, Artificial Sequence
  Synthetic
cttcgatcacacg

SEQ ID NO: 14 - DNA, 13 bp, Artificial Sequence
  Synthetic
atcgcacacgccc

SEQ ID NO: 15 - DNA, 28 bp, Artificial Sequence
  Synthetic
  misc_feature (22)..(28): n is a or c or g or t
gttctacacgagtcactgcagnnnnnnn

SEQ ID NO: 16 - DNA, 19 bp, Artificial Sequence
  Synthetic
gttctacacgagtcactgc

SEQ ID NO: 17 - DNA, 13 bp, Artificial Sequence
  Synthetic
ccgtatcggttcc

SEQ ID NO: 18 - DNA, 13 bp, Artificial Sequence
  Synthetic
ccaagacccgtac

SEQ ID NO: 19 - DNA, 13 bp, Artificial Sequence
  Synthetic
taataggaacgcg

SEQ ID NO: 20 - DNA, 13 bp, Artificial Sequence
  Synthetic
atgtagtcgccgt

SEQ ID NO: 21 - DNA, 13 bp, Artificial Sequence
  Synthetic
tttcgtagcgtgc

SEQ ID NO: 22 - DNA, 13 bp, Artificial Sequence
  Synthetic
atactggcgagta

SEQ ID NO: 23 - DNA, 13 bp, Artificial Sequence
  Synthetic
cggcgccggacaa

SEQ ID NO: 24 - DNA, 13 bp, Artificial Sequence
  Synthetic
ctcgtcgacccac

SEQ ID NO: 25 - DNA, 13 bp, Artificial Sequence
  Synthetic
cgcgcggttagca

SEQ ID NO: 26 - DNA, 13 bp, Artificial Sequence
  Synthetic
cgcggttagcatg

SEQ ID NO: 27 - DNA, 13 bp, Artificial Sequence
  Synthetic
taagagcgcgttc

SEQ ID NO: 28 - DNA, 13 bp, Artificial Sequence
  Synthetic
atcgtagtgtacc

SEQ ID NO: 29 - DNA, 13 bp, Artificial Sequence
  Synthetic
ccgatgtgcgcag

SEQ ID NO: 30 - DNA, 13 bp, Artificial Sequence
  Synthetic
cgtgcccgcgtca

SEQ ID NO: 31 - DNA, 13 bp, Artificial Sequence
  Synthetic
cgcctgcgattat

SEQ ID NO: 32 - DNA, 13 bp, Artificial Sequence
  Synthetic
atctcgatacgat

SEQ ID NO: 33 - DNA, 13 bp, Artificial Sequence
  Synthetic
cgaatcggacgag

SEQ ID NO: 34 - DNA, 13 bp, Artificial Sequence
  Synthetic
aatcggacgagac

SEQ ID NO: 35 - DNA, 13 bp, Artificial Sequence
  Synthetic
actatggtatccg

SEQ ID NO: 36 - DNA, 13 bp, Artificial Sequence
  Synthetic
cgctatacggact

SEQ ID NO: 37 - DNA, 13 bp, Artificial Sequence
  Synthetic
tcgccgccggttc

SEQ ID NO: 38 - DNA, 13 bp, Artificial Sequence
  Synthetic
gccgccggttcta

SEQ ID NO: 39 - DNA, 13 bp, Artificial Sequence
  Synthetic
taaatcacggcgg

SEQ ID NO: 40 - DNA, 13 bp, Artificial Sequence
  Synthetic
gcgcgcttagcta

SEQ ID NO: 41 - DNA, 13 bp, Artificial Sequence
  Synthetic
gcgcactatcgat

SEQ ID NO: 42 - DNA, 13 bp, Artificial Sequence
  Synthetic
cacgatacggcca

SEQ ID NO: 43 - DNA, 13 bp, Artificial Sequence
  Synthetic
agcgcactatcga

SEQ ID NO: 44 - DNA, 13 bp, Artificial Sequence
  Synthetic
agtccaattcgtg

SEQ ID NO: 45 - DNA, 13 bp, Artificial Sequence
  Synthetic
gtcggcgaccctt

SEQ ID NO: 46 - DNA, 13 bp, Artificial Sequence
  Synthetic
aggacgacgctac

SEQ ID NO: 47 - DNA, 13 bp, Artificial Sequence
  Synthetic
tcgtctggtacga

SEQ ID NO: 48 - DNA, 13 bp, Artificial Sequence
  Synthetic
taaagggacgcgc

SEQ ID NO: 49 - DNA, 13 bp, Artificial Sequence
  Synthetic
cgtggttcgcggc

SEQ ID NO: 50 - DNA, 13 bp, Artificial Sequence
  Synthetic
tcctaacgcgccg

SEQ ID NO: 51 - DNA, 13 bp, Artificial Sequence
  Synthetic
cagtcgctcggtt

SEQ ID NO: 52 - DNA, 13 bp, Artificial Sequence
  Synthetic
ccgtggttcgcgg

SEQ ID NO: 53 - DNA, 13 bp, Artificial Sequence
  Synthetic
atgcgcgcatgtc

SEQ ID NO: 54 - DNA, 13 bp, Artificial Sequence
  Synthetic
cgggtccacgact

SEQ ID NO: 55 - DNA, 13 bp, Artificial Sequence
  Synthetic
ctcgcgagtgtac

SEQ ID NO: 56 - DNA, 13 bp, Artificial Sequence
  Synthetic
tcgcgagtgtaca

SEQ ID NO: 57 - DNA, 13 bp, Artificial Sequence
  Synthetic
ttgcgtgacgcag

SEQ ID NO: 58 - DNA, 13 bp, Artificial Sequence
  Synthetic
caacggcgttgct

SEQ ID NO: 59 - DNA, 13 bp, Artificial Sequence
  Synthetic
aacggcgttgcta

SEQ ID NO: 60 - DNA, 13 bp, Artificial Sequence
  Synthetic
gcgttgctacacg

SEQ ID NO: 61 - DNA, 13 bp, Artificial Sequence
  Synthetic
acgagtcgtcgat

SEQ ID NO: 62 - DNA, 13 bp, Artificial Sequence
  Synthetic
cggctaaacccgc

SEQ ID NO: 63 - DNA, 13 bp, Artificial Sequence
  Synthetic
gcggctcgtgcgt

SEQ ID NO: 64 - DNA, 13 bp, Artificial Sequence
  Synthetic
acgagtcgtcgat

SEQ ID NO: 65 - DNA, 13 bp, Artificial Sequence
  Synthetic
cggctaaacccgc

SEQ ID NO: 66 - DNA, 13 bp, Artificial Sequence
  Synthetic
gcggctcgtgcgt

SEQ ID NO: 67 - DNA, 13 bp, Artificial Sequence
  Synthetic
cactaactcgcgc

SEQ ID NO: 68 - DNA, 13 bp, Artificial Sequence
  Synthetic
cactaactcgcgc

SEQ ID NO: 69 - DNA, 13 bp, Artificial Sequence
  Synthetic
actaactcgcgcc

SEQ ID NO: 70 - DNA, 13 bp, Artificial Sequence
  Synthetic
actaactcgcgcc

SEQ ID NO: 71 - DNA, 13 bp, Artificial Sequence
  Synthetic
tcacgtacaccgc

SEQ ID NO: 72 - DNA, 13 bp, Artificial Sequence
  Synthetic
acgtacaccgcag

SEQ ID NO: 73 - DNA, 13 bp, Artificial Sequence
  Synthetic
cggctacggagat

SEQ ID NO: 74 - DNA, 13 bp, Artificial Sequence
  Synthetic
acgcaaaggcgac

SEQ ID NO: 75 - DNA, 13 bp, Artificial Sequence
  Synthetic
ctcttagccggtt

SEQ ID NO: 76 - DNA, 13 bp, Artificial Sequence
  Synthetic
tagccggttaggg

SEQ ID NO: 77 - DNA, 13 bp, Artificial Sequence
  Synthetic
cccgccgacggtc

SEQ ID NO: 78 - DNA, 13 bp, Artificial Sequence
  Synthetic
ccgacggtctcgc

SEQ ID NO: 79 - DNA, 13 bp, Artificial Sequence
  Synthetic
cagtgaaacgacg

SEQ ID NO: 80 - DNA, 13 bp, Artificial Sequence
  Synthetic
cagcggcgatcgg

SEQ ID NO: 81 - DNA, 13 bp, Artificial Sequence
  Synthetic
gaacggagcgcat

SEQ ID NO: 82 - DNA, 13 bp, Artificial Sequence
  Synthetic
gcttaggcgctta

SEQ ID NO: 83 - DNA, 13 bp, Artificial Sequence
  Synthetic
gtcgagcctcgag

SEQ ID NO: 84 - DNA, 13 bp, Artificial Sequence
  Synthetic
tcgagcctcgagt

SEQ ID NO: 85 - DNA, 13 bp, Artificial Sequence
  Synthetic
cggacggcatttc

SEQ ID NO: 86 - DNA, 13 bp, Artificial Sequence
  Synthetic
tataacgtacgaa

SEQ ID NO: 87 - DNA, 13 bp, Artificial Sequence
  Synthetic
aacgtacgaagtc

SEQ ID NO: 88 - DNA, 13 bp, Artificial Sequence
  Synthetic
gtactatcgaacg

SEQ ID NO: 89 - DNA, 13 bp, Artificial Sequence
  Synthetic
ttagtcggcttcg

SEQ ID NO: 90 - DNA, 13 bp, Artificial Sequence
  Synthetic
cacgcgatgcaac

SEQ ID NO: 91 - DNA, 13 bp, Artificial Sequence
  Synthetic
tttgcggcgtttc

SEQ ID NO: 92 - DNA, 13 bp, Artificial Sequence
  Synthetic
acacggtcggtaa

SEQ ID NO: 93 - DNA, 13 bp, Artificial Sequence
  Synthetic
cgttcgaacgtgg

SEQ ID NO: 94 - DNA, 13 bp, Artificial Sequence
  Synthetic
tcgaacgtggcga

SEQ ID NO: 95 - DNA, 13 bp, Artificial Sequence
  Synthetic
accgccctcgcat

SEQ ID NO: 96 - DNA, 13 bp, Artificial Sequence
  Synthetic
cgaataccgccct

SEQ ID NO: 97 - DNA, 13 bp, Artificial Sequence
  Synthetic
cgaccatgcggct

SEQ ID NO: 98 - DNA, 13 bp, Artificial Sequence
  Synthetic
acgcaacgcctcc

SEQ ID NO: 99 - DNA, 13 bp, Artificial Sequence
  Synthetic
cgagcttcgtgcg

SEQ ID NO: 100 - DNA, 13 bp, Artificial Sequence
  Synthetic
cgtgtagctcgat

SEQ ID NO: 101 - DNA, 13 bp, Artificial Sequence
  Synthetic
gagtgatacgcgg

SEQ ID NO: 102 - DNA, 13 bp, Artificial Sequence
  Synthetic
gtgatacgcggac

SEQ ID NO: 103 - DNA, 13 bp, Artificial Sequence
  Synthetic
tgatacgcggaca

SEQ ID NO: 104 - DNA, 13 bp, Artificial Sequence
  Synthetic
gatacgcggacaa

SEQ ID NO: 105 - DNA, 13 bp, Artificial Sequence
  Synthetic
tcgcacagacgtt

SEQ ID NO: 106 - DNA, 13 bp, Artificial Sequence
  Synthetic
cggcggcgatctt

SEQ ID NO: 107 - DNA, 13 bp, Artificial Sequence
  Synthetic
atcgcacacgccc

SEQ ID NO: 108 - DNA, 13 bp, Artificial Sequence
  Synthetic
cttcgatcacacg

SEQ ID NO: 109 - DNA, 13 bp, Artificial Sequence
  Synthetic
gccgaacccgaga

SEQ ID NO: 110 - DNA, 13 bp, Artificial Sequence
  Synthetic
cacgagtcggtgc

SEQ ID NO: 111 - DNA, 13 bp, Artificial Sequence
  Synthetic
tacgcccaggtat

SEQ ID NO: 112 - DNA, 13 bp, Artificial Sequence
  Synthetic
atcgggacccgat

SEQ ID NO: 113 - DNA, 13 bp, Artificial Sequence
  Synthetic
atcgagggttacc

SEQ ID NO: 114 - DNA, 13 bp, Artificial Sequence
  Synthetic
tcgccgtccacga

SEQ ID NO: 115 - DNA, 13 bp, Artificial Sequence
  Synthetic
ctgtcgtgtgcgg

SEQ ID NO: 116 - DNA, 13 bp, Artificial Sequence
  Synthetic
atgcgtgaagtcg

SEQ ID NO: 117 - DNA, 13 bp, Artificial Sequence
  Synthetic
atgactccgacgt

SEQ ID NO: 118 - DNA, 13 bp, Artificial Sequence
  Synthetic
cggttgaataagc

SEQ ID NO: 119 - DNA, 13 bp, Artificial Sequence
  Synthetic
acgctgaaatcgg

SEQ ID NO: 120 - DNA, 13 bp, Artificial Sequence
  Synthetic
gtacgcgggctta

SEQ ID NO: 121 - DNA, 13 bp, Artificial Sequence
  Synthetic
tatcggacaaggc

SEQ ID NO: 122 - DNA, 13 bp, Artificial Sequence
  Synthetic
atcatgaacgacg

SEQ ID NO: 123 - DNA, 13 bp, Artificial Sequence
  Synthetic
attgtcaacgacc

SEQ ID NO: 124 - DNA, 13 bp, Artificial Sequence
  Synthetic
tagggttaccgcc

SEQ ID NO: 125 - DNA, 13 bp, Artificial Sequence
  Synthetic
aggtagtcgccta

SEQ ID NO: 126 - DNA, 13 bp, Artificial Sequence
  Synthetic
tcgacaacgtttc

SEQ ID NO: 127 - DNA, 13 bp, Artificial Sequence
  Synthetic
tcgaacactggta

SEQ ID NO: 128 - DNA, 13 bp, Artificial Sequence
  Synthetic
gctcgttatagat

SEQ ID NO: 129 - DNA, 13 bp, Artificial Sequence
  Synthetic
ttcgaattgaccg

SEQ ID NO: 130 - DNA, 13 bp, Artificial Sequence
  Synthetic
cgaattgaccgta

SEQ ID NO: 131 - DNA, 13 bp, Artificial Sequence
  Synthetic
tcgtgcttcgggc

SEQ ID NO: 132 - DNA, 13 bp, Artificial Sequence
  Synthetic
cggagtctacggg

SEQ ID NO: 133 - DNA, 13 bp, Artificial Sequence
  Synthetic
gcggttaactagt

SEQ ID NO: 134 - DNA, 13 bp, Artificial Sequence
  Synthetic
cgattgccaaacg

SEQ ID NO: 135 - DNA, 13 bp, Artificial Sequence
  Synthetic
gatgtcgataacc

SEQ ID NO: 136 - DNA, 13 bp, Artificial Sequence
  Synthetic
aatcgtctatgcg

SEQ ID NO: 137 - DNA, 13 bp, Artificial Sequence
  Synthetic
atcgtctatgcgc

SEQ ID NO: 138 - DNA, 13 bp, Artificial Sequence
  Synthetic
cgccgtatgcaac

SEQ ID NO: 139 - DNA, 13 bp, Artificial Sequence
  Synthetic
gccttaatccgct

(19) United States
(12) Patent Application Publication
(10) Pub. No.: US 2018/0044724 A1
(43) Pub. Date: Feb. 15, 2018

(54) DNA LIGASE MEDIATED DNA AMPLIFICATION

(71) Applicant: AnchorDx Medical Co.,Ltd., Guangzhou (CN)

(72) Inventors: Jian-Bing FAN, Guangzhou (CN); Yangbin GAO, Renshou County (CN); Weihong XU, Guangzhou (CN)

(21) Appl. No.: 15/476,979

(22) Filed: Apr. 1, 2017

(30) Foreign Application Priority Data
Apr. 1, 2016 (CN) 201610206799.9

(51) Int. Cl. C12Q 1/68

(52) U.S. Cl. CPC C12Q 1/6858; C12Q 1/686; C12Q 1/6855

(57) ABSTRACT

The disclosure provides methods of DNA amplification mediated by DNA ligase. Specifically disclosed is a method of amplifying a target region at DNA level, which comprises repeating cycles of amplification comprising the following steps: denaturing DNA template to obtain single target DNA strands; hybridizing a primer pair comprising an upstream primer and a downstream primer to the target DNA strand, wherein the upstream primer is hybridized to a first nucleic acid sequence of the target region, and the downstream primer is hybridized to a second nucleic acid sequence of the target region; the first nucleic acid sequence is downstream to the second nucleic acid sequence on the target DNA strand, with the downstream primer containing a phosphorylated 5′ end; ligating the upstream primer or extension product thereof to the downstream primer or extension product thereof, to obtain a semi-amplification product. This disclosure also discloses a kit used to amplify a target region at DNA level.

### CROSS REFERENCE TO RELATED APPLICATIONS

This application claims priority to Chinese Patent Application No. 201610206799.9, filed on Apr. 1, 2016, the disclosure of which is hereby incorporated by reference in its entirety.

### FIELD OF THE INVENTION

This invention relates to DNA ligase mediated amplification methods, particularly relates to methods of DNA ligase mediated amplification of low-content DNA. Such amplification technique can be applied in detection of SNP or gene copy number.

### BACKGROUND OF THE INVENTION

In human and other mammals, many diseases are closely related to gene mutation. There are many forms of mutation. For example, in a single individual or cell, the most common type of mutation typically occurs at a single site of a DNA sequence (Single Nucleotide Polymorphism, SNP); yet another common type of mutation is copy number variation (CNV).

SNP mainly refers to polymorphism of DNA sequence at genome level caused by mutation of single nucleotide. The polymorphism presented by SNP only involves mutation of single base. Such mutation may be caused by transition or transversion of a single base, and also may be caused by insertion or deletion of a base, but the commonly known SNP does not include the latter two circumstances. In human genome, frequency of single nucleotide mutation is about 0.1%, i.e., roughly there is one SNP in every 1000 bases. Studies show that SNP may be used to discover diseases related gene mutations. Some SNPs do not directly cause diseases, but become important markers for certain diseases because their locations are adjacent to certain disease-related genes.

CNV usually refers to the increase or decrease in copy numbers of having a length of more than 1kb, which is mainly characterized by microdeletions and/or microduplications. CNV is a major class of structural variation (SV). The mutation rate of CNV sites is significantly higher than that of SNP, and CNV is one of the important pathogenic factors of human diseases. CNV may cause Mendelian monogenetic and rare diseases, and is also related to complicated diseases. For example, numbers of studies show that CNV exists in tumors of different types. Possible pathogenic mechanisms of CNV include gene dosage effect, gene disruption, gene fusion, and position effect, etc. Intensive studies on CNV can expand our understanding towards composition of human genome, inter-individual genetic differences, and genetic pathogenic factors. Detection on CNV may also be helpful for accurately determining individual disease type, subtype or drug resistance, and thereby designing specific therapeutic regimens with pertinence.

Currently, one powerful tool for exploring SNP and CNV is single cell sequencing. Single cell sequencing refers to a gene sequencing technique at single-cell level (not restricted to one cell, but cells in relatively small amount (e.g., less than 10³ cells), DNA with an amount equivalent to the amount of DNA in a single-cell level (e.g., single chromosome or 0.5-3 pg genomic DNA)). DNA or RNA in a single cell is merely at picrogram (pg) level, which is far below the required minimum loading amount for existing sequencing machines, therefore, trace amount of nucleic acid molecules at single-cell level must be amplified first before used for sequencing. Gene amplification at single cell level may bring some problems: i.) Existing technique for single cell transcriptomes amplification can hardly obtain full-length transcripts. ii.) Bias exists in the amplification, i.e., some transcripts or certain regions are more easily amplified, therefore the quantitative relationship between the final expression levels of the genes may not truly reflect the real condition within the cells. iii.) Errors may be introduced during the amplification process, and since the amount of the template is extremely low, said errors may be amplified by many folds. iv.) Biases happened at the 3′ and 5′ end in the amplification process can cause problems in the calculation of expression level, while regarding SNP and CNV, biases in the amplification process can greatly affect accuracy and resolution of SNP and CNV identification.

Therefore, a method of decreasing errors introduced during amplification to the most extent and/or effectively eliminating amplification biases is needed.

### BRIEF SUMMARY OF THE INVENTION

One aspect of this disclosure provides a method of amplifying a target region at DNA level, particularly a method of amplifying a target region in low-content DNA.

In some embodiments, the method of amplifying a target region at DNA level comprises: repeating N cycles of first round amplification, wherein N is an integer and the first round amplification comprising the following steps: i) denaturing DNA template to obtain a single target DNA strand; ii) hybridizing first round amplification primer pairs to said target DNA strand to amplify the target region, each said first round amplification primer pair comprising an upstream primer and a downstream primer, the upstream primer is hybridized to a first nucleic acid sequence of the target region, the downstream primer is hybridized to a second nucleic acid sequence of the target region, wherein the first nucleic acid sequence and the second nucleic acid sequence are on the same target DNA strand, and wherein there are m nucleotides between the first nucleic acid sequence and the second nucleic acid sequence, and m is an integer ≧0, wherein the first nucleic acid sequence is downstream to the second nucleic acid sequence on the target DNA strand, and the downstream primer contains a phosphorylated 5′ end; iii) optionally, extending at 3′ end of the upstream primer and/or the downstream primer using the antisense strand as template; iv) ligating the upstream primer or extension product thereof to the downstream primer or extension product thereof to obtain a semi-amplification product comprising a target region, wherein the semi-amplification product is an antisense strand of the single target DNA strand. In some other embodiments, the initial DNA template is double-strand DNA. In some embodiments, the initial DNA template is single-strand DNA.

In some embodiments, the length of the first nucleic acid sequence and the second nucleic acid sequence is no less than 6 bp, no less than 7 bp, no less than 8 bp, no less than 9 bp, no less than 10 bp, no more than 50 bp, no more than 40 bp, no more than 30 bp, 6-50 bp, 6-40 bp, 6-30 bp, 6-20 bp respectively. In some embodiments, the length of the first nucleic acid sequence and the second nucleic acid sequence is 6-30 bp respectively.

In some embodiments, m=0, i.e., the first nucleic acid sequence is immediately adjacent to the second nucleic acid sequence. In some embodiments, m≦100, i.e., the interval between the first nucleic acid sequence and the second nucleic acid sequence is no more than 100 bp. In some embodiments, the interval between the first nucleic acid sequence and the second nucleic acid sequence is no more than 90 bp, no more than 80 bp, no more than 70 bp, no more than 60 bp, no more than 50 bp, no more than 40 bp, no more than 30 bp, no more than 20 bp, no more than 10 bp. In some other embodiments, the interval between the first nucleic acid sequence and the second nucleic acid sequence is no less than 90 bp, no less than 80 bp, no less than 70 bp, no less than 60 bp, no less than 50 bp, no less than 40 bp, no less than 30 bp, no less than 20 bp, no less than 10 bp.

In some embodiments, the upstream primer comprises adapter sequence. In some embodiments, the upstream primer and the downstream primer both comprise adapter sequences. In some embodiments, the upstream primer and the downstream primer comprise hybrid sequences. In some specific embodiments, the hybrid sequence further comprise random sequence. In some embodiments, the hybrid sequence comprised in the upstream primer comprises or is random sequence, and the hybrid sequence comprised in the downstream primer is hybrid sequence that specifically recognizes the target region. In some embodiments, the hybrid sequences comprised in the upstream primer and the downstream primer are both hybrid sequences that specifically recognize the target region.

In some embodiments, the upstream primer comprises adapter sequence at its 5′ end. In some embodiments, the upstream primer comprises adapter sequence at its 5′ end, and the downstream primer comprises adapter sequence at its 3′ end.

In some embodiments, the first nucleic acid sequence is immediately adjacent to the second nucleic acid sequence, and the 3′ end of the upstream primer and the 5′ end of the downstream primer or extension product thereof are directly ligated via a heat resistant ligase to obtain semi-amplification product.

In some embodiments, there are m nucleotides between the first nucleic acid sequence and the second nucleic acid sequence, and the upstream primer is extended at the 3′ end via DNA polymerase by m nucleotides to the site immediately adjacent to the 5′ end of the downstream primer, to obtain an extension product of the upstream primer. In some embodiments, extension product of the upstream primer is ligated to the downstream primer or extension product thereof via a heat resistant ligase, to obtain a semi-amplification product.

In some embodiments, the first round amplification is linear amplification, and the semi-amplification product is the first round amplification product. The method of amplifying a target region at DNA level further comprises: sequencing the semi-amplification product to determine the sequence of the target region.

In some embodiments, the hybrid sequence comprised in the upstream primer is a random sequence, and the first round amplification further comprises: v) hybridizing the upstream primer to the semi-amplification product, and extending at 3′ end of the upstream primer using the semi-amplification product as template to obtain a first round amplification product.

In some embodiments, the method of amplifying a target region at DNA level further comprises: using the first round amplification product as template, amplifying the target region through polymerase chain reaction (PCR), to generate exponential amplification product. In some embodiments, the method of amplifying a target region at DNA level further comprises: sequencing the exponential amplification product to determine sequence of the target region.

In some embodiments, the method of amplifying a target region at DNA level further comprises: comparing sequencing result of the target sequence with a reference sample to determine copy number of the target sequence. In some embodiments, the reference sample has two copies.

Another aspect of this disclosure provides a kit used to amplify a target region at DNA level. In some embodiments, the kit comprises: first round amplification primer pairs, wherein each said primer pair comprises an upstream primer and a downstream primer, the upstream primer and downstream primer are hybridized to target DNA strand, wherein the upstream primer is hybridized to the first nucleic acid sequence of the target region, and the downstream primer is hybridized to the second nucleic acid sequence of the target region, wherein there are m nucleotides between the first nucleic acid sequence and the second nucleic acid sequence, and m is an integer ≧0, wherein the first nucleic acid sequence is downstream to the second nucleic acid sequence on the target DNA strand, and the downstream primer contains a phosphorylated 5′ end; and a ligating reagent, wherein the ligating reagent is used to ligate the upstream primer or extension product thereof to the downstream primer or extension product thereof to obtain an semi-amplification product.

In some embodiments, the ligating reagent comprises a ligase and a ligation reaction agent. In some embodiments, the ligase is a heat resistant ligase.

In some embodiments, the kit used to amplify a target region at DNA level further comprises: an extension reagent, the extension reagent is used to extend at 3′ end of the upstream primer and/or the downstream primer, using the antisense strand as template, to obtain an extension product of the upstream primer and/or downstream primer. In some embodiments, the extension reagent comprises a DNA polymerase, an extension reaction agent, and dNTP, the dNTP consists of any one or more of dATP, dTTP, dGTP, and dCTP. In some embodiments, the extension reagent may also be used to extend at 3′ of the upstream primer, using the semi-amplification product as template, to obtain the first round amplification product, when the upstream primer is hybridized to the semi-amplification product.

In some embodiments, the upstream primer comprises 5′ end adapter sequence. In some embodiments, the upstream primer comprises 5′ end adapter sequence, and the downstream primer comprises 3′ end adapter sequence. In some embodiments, the upstream primer and the downstream primer comprise hybrid sequences. In some specific embodiments, the hybrid sequence further comprises random sequence. In some embodiments, the hybrid sequence comprised in the upstream primer comprises or is random sequence, and the hybrid sequence comprised in the downstream primer is hybrid sequence that specifically recognizes the target region. In some embodiments, the hybrid sequences comprised in both the upstream primer and the downstream primer are hybrid sequences that specifically recognize the target region. In some embodiments, the kit used to amplify a target region at DNA level further comprises: an exponential amplification reagent, the exponential amplification reagent is used to amplify the target region using the first round amplification product as template, to obtain exponential amplification product. In some embodiments, the exponential amplification reagent comprises DNA polymerases, an exponential amplification reaction agent, and dNTP.

In some embodiments, the exponential amplification reagent further comprises a universal primer for exponential amplifications, wherein the universal primer for exponential amplifications contains a sequence identical to or reversely complementary to the 5′ end adapter sequence of the upstream primer, and/or a sequence identical to or reversely complementary to the 3′ end adapter sequence of the downstream primer.

In some embodiments, the kit used to amplify a target region at DNA level further comprises: a sequencing reagent, the sequencing reagent is used to sequence the first round amplification product or the exponential amplification product.

### BRIEF DESCRIPTION OF THE DRAWINGS

The aforesaid and other features of the subject matter of the present disclosure are more adequately described through a combination of the drawings with the following description and attached claims. It can be understood, that these drawings only displays several embodiments of the subject matter of the present disclosure, and thus should not be regarded as limitation on the scope of the subject matter of the present disclosure. Through the adoption of the drawings, the subject matter of the present disclosure shall be illustrated more explicitly and in more details.

FIG. 1 is a schematic for the fundamental principle of the amplification method of the present disclosure where the first round amplification is a linear amplification;

FIG. 2 is a schematic for the fundamental principle of the amplification method of the present disclosure where the first round amplification is a not linear amplification;

FIG. 3 is a schematic for the fundamental principle of the exponential amplification in the amplification method of the present disclosure;

FIG. 4 is a schematic for the fundamental principle of use of an exemplary amplification method of the present disclosure in detecting DNA copy number variation (CNV);

FIG. 5 is a detailed embodiment of the amplification method of the present disclosure.

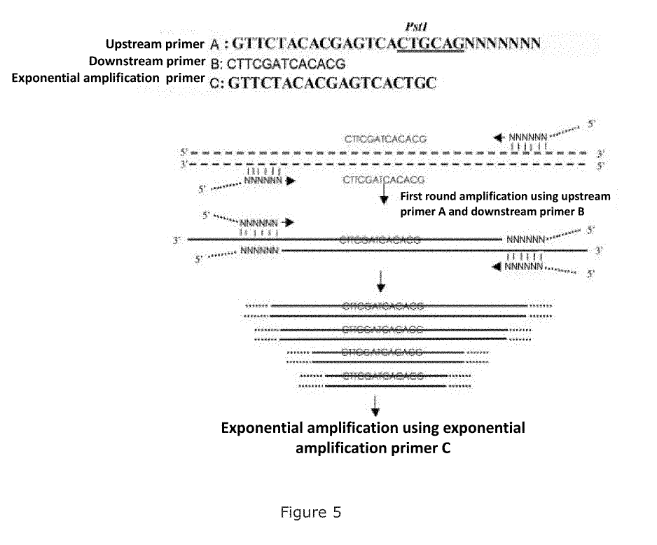

### DETAILED DESCRIPTION OF ILLUSTRATIVE EMBODIMENTS

One aspect of this disclosure provides methods of amplifying a target region at DNA level, particularly a method of amplifying a target region in low-content DNA. This disclosure is at least partly based on the following discoveries, i.e., methods of DNA ligase mediated DNA amplification can achieve one or both of the following effects: 1) decrease of errors introduced in the amplification process; 2) effective elimination of amplification biases.

In some embodiments, the method provided by the present disclosure of amplifying a target region at DNA level comprises: repeating N cycles of the first round amplification, wherein N is an integer and 1≦N≦40, the first round amplification comprising the following steps: i) denaturing DNA template to obtain a single target DNA strand; ii) hybridizing a primer pair used to amplify the target region to said single target DNA strand, each primer pair comprises an upstream primer and a downstream primer, the upstream primer is hybridized to a first nucleic acid sequence of the target region, the downstream primer is hybridized to a second nucleic acid sequence of the target region, wherein there are m nucleotides between the first nucleic acid sequence and the second nucleic acid sequence, and m is an integer ≧0, wherein the first nucleic acid sequence is downstream to the second nucleic acid sequence on the target DNA strand, and the downstream primer contains a phosphorylated 5′ end; iii) optionally, extending at 3′ end of the upstream primer and/or downstream primer using the antisense strand as template; iv) obtaining a semi-amplification product by ligating the upstream primer or extension product thereof, to the downstream primer or extension product thereof.

The term “DNA” as used in the present disclosure refers to the long chain polymer biological macromolecule carrying genetic instructions, consisting of deoxyribonucleotides. “DNA level” refers to nucleotide level. Every nucleotide in DNA consists of a nitrogenous base, a five-carbon sugar (2-deoxyribose) and phosphate groups. Neighboring nucleotides are linked via ester bonds formed by deoxyribose and phosphoric acid, thereby forming a long chain framework. Two ends of a nucleotide molecule are asymmetrical, containing a phosphate group and a hydroxyl group respectively. Neighboring nucleotide molecules in a DNA strand forms phosphodiester bonds with each other. Molecules at the ends of the DNA strand retains a phosphate group and a hydroxyl group respectively, wherein the end containing phosphate group is known as the 5′ end, and the end containing hydroxyl group is known as the 3′ end. The location of one certain DNA fragment/base “a” relative to another fragment/base “b” on the same DNA strand can be expressed as upstream or downstream. Upstream and downstream are relative concepts. When describing that DNA fragment/base “a” is upstream to fragment/base “b”, it refers to that, relative to fragment/base “b”, DNA fragment/base “a” is closer to the 5′ end of the DNA strand where it locates on. Conversely, when describing that DNA fragment/base “A” is downstream to fragment/base “B”, it refers to that, relative to fragment/base “B”, DNA fragment/base “A” is closer to the 3′ end of the DNA strand where it locates on.

Generally there are four types of nitrogenous bases in DNA nucleotides, namely adenine (A), guanine (G), and cytosine (C), thymine (T). The bases on the two DNA long chains pair via hydrogen bonds, wherein adenine (A) pairs with thymine (T), and guanine (G) pairs with cytosine (C), thereby allowing most DNA to exist in a double-strand, double-helix structure. Hydrogen bonds of the double-strand structure may break upon heat or alkali treatment, and double-strand DNA molecules are thereby denatured and separated into two single-strand DNA.

The term “target region” as used in the present disclosure refers generally to all target locations for detection. In some preferred embodiments, the target regions are known CNV or SNP regions related to certain diseases (e.g., tumor, inflammation, birth defect, etc.). In some preferred embodiments, the target regions are genome regions related to chromosomal microdeletion and microduplication syndromes (MMS).

In the first cycle of the first round amplification, the term “DNA template” as used herein refers to the initial DNA template, whereas in the Nth cycle where N>1, the term “DNA template” refers to the double-strand template contained in the amplification product (semi-amplification product and/or the first round amplification product) obtained in step iii) of the N-1th cycle.

The term “initial DNA template” as used in the present disclosure refers to the DNA sample initially used as template in the amplification method of the present disclosure. The initial DNA template used in amplification in the present disclosure can be single-strand DNA, as well as double-strand DNA.

The initial DNA template can derive from biological samples of any form. The term “biological samples” as used in the present disclosure includes, but is not limited to cells (including but not limited to: bacteria, virus or animal and plant cells), tissues (including but not limited to: normal, necrotic, cancerous, para-cancerous tissues, etc.), body fluid (including but not limited to: blood, blood plasma, blood serum, saliva, amniocentesis fluid, pleural effusion, ascites), etc. Biological samples involved in the present disclosure can be from any species or biological species, including but not limited to human, mammal, ox, pig, sheep, horse, rodent, poultry, fish, zebra fish, shrimp, plant, yeast, virus or bacteria. A person skilled in the art can obtain the biological samples by any means known in the art, including but not limited to by sampling methods through cell culture, operation, anatomy, blood drawing, wiping, and lavage, etc. Biological samples can be provided in any appropriate form, e.g., can be provided in freshly isolated, paraffin embedded, refrigerated, and frozen forms, etc.

In some embodiments, the biological samples are cells, the initial DNA template is genome DNA. In some embodiments, the initial DNA template derives from a single cell. In some embodiments, the initial DNA template derives from multiple cells, e.g., derives from two or more cells of the same type. The term “multiple cells” as used in the present disclosure refers to no more than 10³, no more than 10², no more than 10 or more than 10³ cells. The above single cells or multiple cells can be from, e.g., preimplantation embryos, embryonic cells in peripheral blood of pregnant women, single sperms, ova, zygotes, cancer cells, bacterial cells, circulating tumor cells, tumor tissue cells, or single or multiple cells of the same type obtained from any tissue. Single cells can be obtained by any means known in the art, including but not limited to, flow cytometry sorting, fluorescent activated cell sorting, magnetic beads separation, semi-automatic cell sorting machines, etc. In some embodiments, cells of specific types can be selected according to different features of single cells, e.g., cells expressing certain specific biological marks.

In some other embodiments, the biological sample is a body fluid. In some embodiments, the initial DNA template derives from blood, blood serum, blood plasma or amniotic fluid. In some embodiments, the initial DNA template is cell-free DNA. “Cell-free DNA” refers to DNA free from cells found in circulatory system (e.g., blood), the source of which is generally believed to be genome DNA released due to apoptosis. Studies show that the size of most cell-free DNA in human body is about 160 bp (see Fan et al., (2010) Analysis of the Size Distributions of Fetal and Maternal Cell-Free DNA by Paired-End Sequencing, Clin Chem 56:8 1279-86). In some embodiments, the cell-free DNA contains circulating tumor DNA. “Circulating tumor DNA” refers to the cell-free DNA derived from tumor cells. In human body, a tumor cell may release its genome DNA into the blood due to causes such as apoptosis and immune reactions. Since a normal cell may also release its genome DNA into the blood, circulating tumor DNA usually consists only a very small part of cell-free DNA. In some embodiments, the initial DNA template is cell-free DNA derived from a pregnant mother, which contains free fetal DNA. “Free fetal DNA” refers to cell-free DNA fragments derived from a fetus and contained in blood of the mother.

A person skilled in the art can obtain the initial template from biological samples by any means known in the art. In some embodiments, the initial DNA template can be ultimately obtained through tissue or cell lysis (e.g., through pyrolysis, alkali lysis, enzyme lysis, mechanical lysis, etc.) and release of nucleic acids within the cells, followed by treatments such as purification. In some specific embodiments, nucleic acids released after lysis can be used as initial DNA template for subsequent amplification even without purification. In some embodiments, the initial DNA template can be obtained through isolating or enriching from blood or blood serum the cell-free DNA contained within.

As previously stated, the method of the present disclosure can be used to amplify certain scarce samples such as low-content initial DNA template, e.g., initial DNA template derived from human ova, germ cells, and in vitro fertilized embryonic cells, etc., or such as circulating tumor DNA, free fetal DNA, etc. The term “low-content initial DNA template” as used in the present disclosure refers to DNA template derived from a single cell, DNA template derived from no more than 10³, no more than 10², no more than 10 or more than 10³ cells, or refers to initial DNA template at an amount equivalent to single cell level, e.g., initial DNA template ≦0.5 pg, ≦3 pg, ≦5 pg, ≦10 pg, ≦50 pg, ≦100 pg, ≦0.5 ng, ≦1 ng, ≦3 ng, and >3 ng.

The term “denaturing DNA template” as used in the present disclosure refers to separating the two strands of double-strand DNA by any means known to a person skilled in the art, including but not limited to thorough pyrolysis (e.g., above 90° C.), alkali (e.g., NaOH) treatment, etc. Where the DNA template is double stranded, single target DNA strands are obtained through the aforesaid denaturing methods. When the initial DNA template is single-strand DNA, denaturing step can be omitted in the first cycle of amplification, and the initial single-strand DNA is the single target DNA strands; or alternatively, the first cycle of amplification may also include denaturing step (e.g., heating until above 90° C. or heating alkali solutions, etc.), but without substantial influence on the initial single-strand DNA template, thus even after the denaturing step, the initial single-strand DNA is still the single target DNA strand. The term “target DNA strand” as used in the present disclosure refers to the strand which the “primer pair” used to amplify the target region can be hybridized to in step ii) of amplification.

The term “primer” as used in the present disclosure refers to a short single-strand DNA fragment, which can be hybridized to a region complementary to it on a DNA or RNA strand and become the starting point of DNA polymerization. DNA polymerase can sequentially add nucleotides complementary to the DNA template strand to the 3′ end of the primer to synthesize a new DNA strand.

The term “complementary to” as used in the present disclosure refers to the ability of a nucleic acid to form hydrogen bonds with another nucleic acid through conventional Watson-Crick base-pairing method or other non-conventional methods. Complementary percentage is used to represent the percentage of the number of residues on one nucleic acid chain molecule which are capable of forming hydrogen bonds (e.g., Watson-Crick base pairs) with the residues on a second nucleic acid sequence. For example, on one nucleic acid chain consisting of 10 nucleic acids, if 5, 6, 7, 8, 9 or 10 nucleic acids can be complementary to residues on a second nucleic acid sequence via hydrogen bonds, the corresponding complementary percentages are 50%, 60%, 70%, 80%, 90% or 100%. “Completely complementary to” refers to that all consecutive residues on one nucleic acid sequence form hydrogen bonds successively with all consecutive residues of the same number on a second nucleic acid sequence. “Substantially complementary to” refers to that in 8, 9, 10, 11, 12, 13, 14, 15, 16, 17, 18, 19, 20, 21, 22, 23, 24, 25, 30, 35, 40, 45, 50 or more nucleic acid regions, there are at least 60%, 65%, 70%, 75%, 80%, 85%, 90%, 95%, 97%, 98%, 99%, or 100% percentage complementary to a second nucleic acid sequence, or refers to two nucleic acid molecules which can hybridize under strict conditions.

The “primer pair” in the first round amplification comprises an upstream primer and a downstream primer, wherein the upstream primer can be hybridized to a first nucleic acid sequence of the target region on a single-strand DNA, and the downstream primer can be hybridized to a second nucleic acid sequence of the target region on the single-strand DNA, wherein the first nucleic acid sequence is downstream to the second nucleic acid sequence on the single DNA strand containing the target region. “Hybridize” refers to that two DNA strands containing complementary base sequences can pair by forming hydrogen bonds between the complementary base sequences, thereby forming a stable double stranded region. That is, the upstream primer can pair with the first nucleic acid sequence of the target region by forming hydrogen bonds to form a stable double stranded region, and the downstream primer can pair with the second nucleic acid sequence of the target region by forming hydrogen bonds to form a stable double stranded region. The upstream primer and the downstream primer may contain any nucleotide that can base-pair with natural nucleic acid, including but not limited to the four natural bases A, T, G, and C, and other nucleotide analogs and modified nucleotides, etc. known by a person skilled in the art, as long as they can pair with the first or second nucleic acid sequence on the target region and enable the amplification reaction.

In some embodiments, the upstream primer and/or the downstream primer contains an adaptor sequence respectively. The adapter sequence in the present disclosure refers to the specific sequence at the 5′ end of the upstream primer and/or the 3′ end of the downstream primer, the length of which can be 8-40 bp, 8-32 bp, 10-30 bp, 12-28 bp, 15-25 bp, 18-22 bp, and 20-24 bp. The adapter sequence contained in the upstream and downstream primers can be identical or different. In the present disclosure, proper adapter sequences are adopted, so that the adapter sequences do not bind to the target region, and self-polymerization of the upstream and downstream primers and polymerization between the upstream and downstream primers can be avoided. In some embodiments, the amplification primer used in subsequent exponential amplification contains a sequence partly or completely complementary to the upstream and downstream adapter sequences. In some embodiments, adapter sequences are selected so that semi-amplification product/first round amplification product/exponential amplification product can be directly used in sequencing.

In some embodiments, the upstream primer and the downstream primer contain hybrid sequences. Hybrid sequences in the present disclosure refer to specific sequences at the 3′ end of the upstream primer and the 5′ end of the downstream primer, the length of which can be 10-40 bp, 15-35 bp, 18-32 bp, 20-30 bp, 22-28 bp, and 24-26 bp. In some embodiments of the present disclosure, a “hybrid sequence” consists of random sequences. In some embodiments of the present disclosure, a “hybrid sequence” consists of at least 4, at least 5, at least 6, at least 7, or at least 8 consecutive random sequences. In some embodiments of the present disclosure, a “hybrid sequence” consists of fixed sequences. In some embodiments of the present disclosure, a “hybrid sequence” substantially consists of fixed sequences, with random sequences being introduced at one or more base sites of the fixed sequences, the one or more base sites can locate at the 3′ or 5′ end as well as in the middle part of the hybrid sequence, and the one or more base sites can be consecutive or nonconsecutive. When a hybrid sequence consists of or substantially consists of fixed sequences, proper fixed sequences can be selected based on the target region, e.g., selecting sequences complementary to sequences of two adjacent or spaced regions on the sequence of the known target region as the fixed sequences in the primers. Since various mutations or single nucleotide polymorphisms (SNPs) exist on many sites of a genome, when such sites are contained in the region of the first nucleic acid sequence and/or the second nucleic acid sequence of the target region, random sequences can be introduced in the fixed sequenced of the primers so that templates containing various mutations of sites and SNPs are all amplified, and the various mutations of sites and SNPs on different sites can be detected in potential subsequent sequencing steps. In some embodiments of the present disclosure, the upstream primer contains hybrid sequences consisting of consecutive random sequences, and the downstream primer contains hybrid sequences consisting of fixed sequences specifically combining to the target region.

Nucleotide sequence in a “random sequence” may have many possible variations. Introducing a random sequence on a certain specific base site of a primer means that, the primer is actually a mixture of primers, comprising a set of primers containing various nucleotide sequences on the aforesaid specific base sites. Every base site in a random sequence may only contain any two, three, or four nucleotides from A, T, G, and C. Nucleotide types on such base sites can be indicated by degenerate codes, e.g., where a certain base site in a random sequence only contains two nucleotides, A and G, the sequence of the site can be indicated as R (i.e., R=A/G). Other degenerate codes include: Y=C/T, M=A/C, K=G/T, S=C/G, W=A/T, H=A/C/T, B=C/G/T, V=A/C/G, D=A/G/T, N=A/C/G/T. The length of a random sequence can be 1 bp, 2 bp, 3 bp, 4 bp, 5 bp, 6 bp, 7 bp, 8 bp, 9 bp, 10 bp or more than 10 bp. Assuming that the length of a random sequence is 1 bp, and the code for random nucleotides contained on the site of 1 bp is N, the primer containing this random sequence is a mixture of 4 primers. Or assuming that the random sequence is 3 bp, wherein the code for random nucleotides contained on each site is H (3 nucleotides A/C/T), the primer containing the random sequence is a mixture of 3×3×3=27 primers. In some embodiments, to eliminate certain undesired situations or to enhance matching with target DNA region, certain limitations are further added on the basis of the maximum degree of randomness (i.e., all four possibilities of A, T, G and C are included), so to select types of nucleotides contained in the random sequence or at certain sites of the random sequence. E.g., in certain embodiments, the adapter sequences contain large amount of G and T, thus to reduce possibility of complementary paring between hybrid sequences and adapter sequences, it can be selected to exclude C and A from the random sequences, and indicate every site of the random sequences by the code K.

Moreover, the downstream primer in the present disclosure contains a phosphorylated 5′ end, i.e., the 5′ end of the downstream primer hybrid sequence contains a phosphate group. There are m nucleotides between the first nucleic acid sequence which the upstream primer is hybridized to and the second nucleic acid sequence which the downstream primer is hybridized to, wherein m is an integer ≧0; when m=0, the first nucleic acid sequence is adjacent to the second nucleic acid sequence, then 3′ end of the upstream primer and 5′ end of the downstream primer are directly ligated using a DNA ligase to obtain semi-amplification product; or where m>0, there is interval between the first nucleic acid sequence and the second nucleic acid sequence, under such circumstance, in the extension step iii) following step ii) of first round amplification, the upstream primer is extended via DNA polymerase at 3′ end by m nucleotides to where immediately adjacent to 5′ end of the downstream primer, to obtain extension product of the upstream primer, and then extension product of the upstream primer and the downstream primer or extension product thereof are ligated using a DNA ligase to obtain semi-amplification product.

A person skilled in the art can selectively use any DNA polymerase known in the art based on practical situations and extend the upstream primer by any means known in the art, wherein the DNA polymerase includes but is not limited to a proper nucleic acid polymerase comprising, but not limited to: a Taq polymerase, a pfu DNA polymerase, a Phusion® hyper fidelity DNA polymerase, a LongAmp Taq DNA polymerase, a OneTaq DNA polymerase, a TOPOTaq DNA polymerase, etc.; wherein the any means known in the art includes but is not limited to ordinary PCR, rolling circle PCR, inverse PCR, nested PCR, etc.

The term “DNA ligase” as used in the present disclosure refers to an enzyme capable of ligating the rear of the 3′ end and the front of the 5′ end of a DNA via formation of phosphodiester bonds between two DNA molecules.

In some embodiments, the DNA ligase is a T4 DNA ligase, capable of catalyzing binding via phosphodiester bonds between the 5′-phosphate end and the 3′ -hydroxyl end of double-strand DNA or RNA with sticky ends or blunt ends. Such catalysis reaction requires ATP as an assistant factor, while its optimum reaction temperature is about 6° C., and enzymatic activity is lost at above 65° C. T4 DNA ligase can repair nicks in single strands of double-strand DNA, double-strand RNA or DNA/RNA hybrids.

In some embodiments, the DNA ligase is a heat resistant DNA ligase.

In some embodiments, the DNA ligase is a heat resistant double-strand DNA ligase (for example but not limited to Ampligase® DNA Ligase, Epicentre Technologies Corp., and Taq DNA ligase). Ampligase® DNA Ligase is a thermostable ligase, which can catalyze ligating reaction between the 5′-phosphate and 3′-hydroxyl groups of NAD-dependent double-strand DNA, with a half-life of 48 hours at 65° C., and a half-life of more than 1 hour at 95° C. Taq DNA ligase is also an NAD-dependent thermostable ligase, which can catalyze formation of phosphodiester bonds, ligating the 5′-phosphate end and the 3′-hydroxyl end of two oligonucleotide strands hybridized to the same target DNA strand via phosphodiester bonds. This ligating reaction can only occur under the condition that the two oligonucleotide strands completely pair with the target DNA and no gap exists in between, and therefore, it can be used to detect single base substitution. Taq DNA ligase is active at 45° C.-65° C.

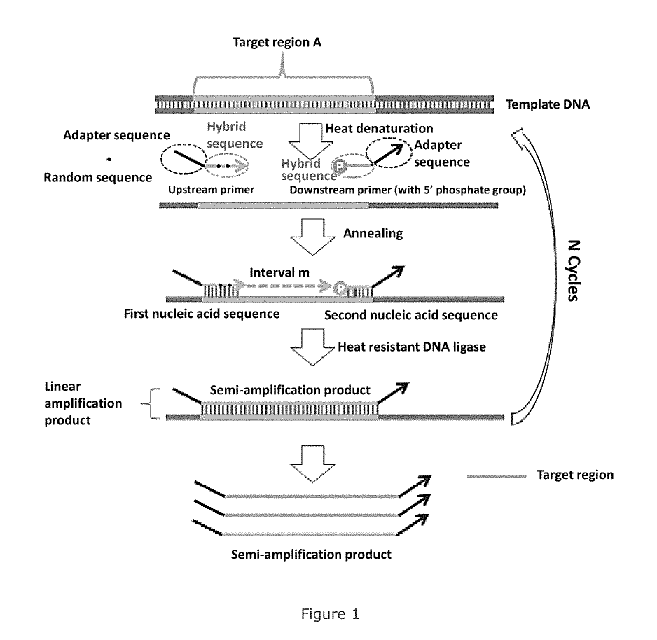

In some other embodiments, the DNA ligase is a heat resistant single-strand DNA ligase (for example but not limited to CircLigase™ ssDNA Ligase, Epicentre Technologies Corp.), which is a thermostable, ATP dependent ligase capable of catalyzing ligation of the 5′-phosphate and 3′-hydroxyl groups of single-strand DNA, and thereby cyclizing the single-strand DNA. CircLigase™ ssDNA Ligase is different from T4 DNA Ligase. T4 DNA Ligase and Ampligase® DNA Ligase can only ligase ends of complementary DNA sequences adjacent to each other, whereas CircLigase™ ssDNA Ligase can ligate ends of single-strand DNA without presence of a reverse complementary sequence. Linear single-strand DNA with more than 15 bases, including cDNA, can all be cyclized by CircLigase. Therefore, this ligase is very import for ligating linear single-strand DNA into cyclic single-strand DNA. Cyclic single-strand DNA molecules can be used as substrates in rolling circle replication and rolling circle transcription studies.

Where both upstream and downstream primers contain adapter sequences, amplification product obtained after the first cycle of linear amplification is a product molecule in which a double stranded region is formed via hydrogen bonds, between a DNA template strand and a DNA strand which has adapter sequences at both ends and a sequence reversely complementary to the target region in the middle (semi-amplification product) (see FIG. 1). It can be understood that where the hybrid sequences of the upstream and downstream primers consist of or substantially consist of sequences reversely complementary to specific regions of the target sequence, since the 5′ end of downstream primer contains an adapter sequence, and the 3′ end contains a phosphate group, the downstream primer cannot extend during amplification, when the upstream primer extends to where immediately adjacent to the 3′ end of the downstream primer, extension product of the upstream primer is ligated to the downstream primer using DNA ligase to obtain a double-strand molecule formed between semi-amplification product and the DNA template strand, wherein of the two strands, the semi-amplification product cannot be used as template for the next cycle of amplification, and in every cycle of amplification, only the initial target DNA strands can be used as template, therefore such means of amplification is called linear amplification. Under such circumstances, after the Nth cycle of linear amplification (wherein N>1), the linear amplification product contains N-1 single-strand semi-amplification products, and one double-strand molecule formed between the semi-amplification product and the DNA template strand. In some embodiments, linear amplification repeats no less than 5, no less than 10, no less than 15, no less than 20, and no less than 30 cycles. In some embodiments, the linear amplification repeats no more than 100, no more than 90, no more than 80, no more than 70, no more than 60, and no more than 50 cycles.

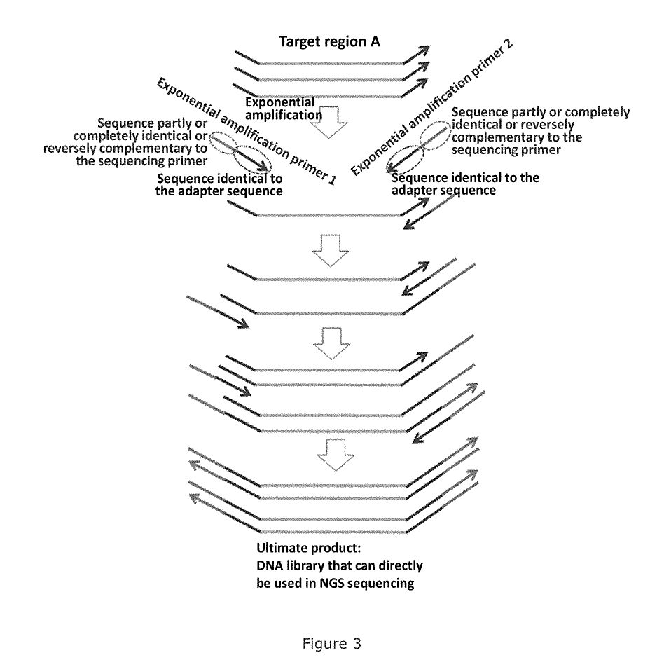

In some embodiments, the upstream primer comprises, from 5′ end to 3′ end, an adapter sequence and a hybrid sequence consisting of consecutive random sequences respectively, and the downstream primer comprises hybrid sequence consisting of or substantially consisting of sequences reversely complementary to specific region of the target sequence, but does not comprise adapter sequences; semi-amplification product obtained in the first cycle of first round amplification, is a DNA strand with adapter sequence at its 5′end and a part or entire of a sequence reversely complementary to the target region in its middle, which binds to the DNA template strand via hydrogen bonds to form a product molecule with double-strand region (see FIG. 3). It can be understood that, contrary to the situation that the downstream primer comprises adapter sequence at its 3′ end, when the hybrid sequence of the upstream primer consists of random sequences and the hybrid sequence of the downstream primer consists of or substantially consists of sequences reversely complementary to specific region of the target sequence, the upstream primer is hybridized to multiple random sites on the DNA template strand and extends downstream, and since the downstream primer does not comprise adapter sequence at its 5′ end, the downstream primer also extends downstream. When the upstream primer is hybridized to upstream of the downstream primer recognition region, it can keep extending downstream until it is immediately adjacent to 3′ end of the downstream primer. The extension product of the upstream primer is then ligated to the downstream primer or extension product thereof using DNA ligase, to obtain a double-strand molecule formed between semi-amplification product and the DNA template strand; since the hybrid sequence of the upstream primer consists of random sequences, it can also recognize sites on semi-amplification product. Therefore using the upstream primer as primer, further amplification can be performed using the semi-amplification product as template for the next round of amplification. Under such circumstances, after multiple cycles of first amplification, the first amplification product comprises multiple single-strand semi-amplification products and a double-strand molecule comprising an adapter sequence of the upstream primer and a sequence complementary to the adapter sequence of the upstream primer respectively at each end.

In some embodiments, the method of amplifying the target region on the initial DNA template provided in the present disclosure further comprises: after N cycles of first round amplification, using a fragment of first round amplification product as template, amplifying the target region through polymerase chain reaction (PCR) to form exponential amplification product. The polymerase chain reaction (PCR) comprises but is not limited to ordinary PCR, rolling circle PCR, inverse PCR, nested PCT, etc. A person skilled in the art can determine reaction conditions such as reaction temperature, reaction program, reaction cycle number, etc. of PCR, based on practical situations. A universal primer for exponential amplification can be used in exponential amplification, which comprises a sequence identical to or reversely complementary to adapter sequence of the upstream primer in the first round amplification (if any) and/or a sequence identical to or reversely complementary to adapter sequences of the downstream primer in the linear amplification (if any).

In some embodiments, the method of amplifying the target region on the initial DNA template provided in the present disclosure comprises sequencing steps, wherein the sequencing steps can be after the first round amplification, i.e., the sequence for sequencing is the first round amplification product; or wherein the sequencing steps can be after the exponential amplification, i.e., the sequence for sequencing is the exponential amplification product. In some specific embodiments, to render the exponential amplification product become a DNA library that can directly be used for sequencing, adapter sequences in the upstream and downstream primers in the first round amplification may be selected to contain a specific sequence identical or reversely complementary to part or entire of the primer used for sequencing, so that the first round amplification product can become a DNA library that can directly be used for sequencing. In some specific embodiments, to render the exponential amplification product become a DNA library that can directly be used for sequencing, the upstream and downstream primers in exponential amplification may further contain at the 5′ ends an adapter sequence required for sequencing, for example but not limited to, specific sequence identical or reversely complementary to part or entire of the primer used for sequencing, or specific sequence identical or reversely complementary to part or entire of the sequence captured on the sequencing board of the platform used for sequencing. In some specific embodiments, to render the exponential amplification product become a DNA library that can directly be used for sequencing, adapter sequences in the upstream and downstream primers in the first round amplification may be selected to contain a specific sequence identical or reversely complementary to part or entire of the primer used for sequencing, and correspondingly, the primers in exponential amplification contain sequences identical or reversely complementary to the adapter sequences of the upstream and downstream primers in the linear amplification, i.e., the primers in exponential amplification contain a specific sequence identical or reversely complementary to part or entire of the primer used for sequencing.

“DNA sequencing library” as the in the present disclosure refers to a collection of DNA fragments with an abundance enough to be sequenced, wherein one end or both ends of each DNA fragment in the collection of DNA fragments contain a specific sequence partly or completely reversely complementary to the primer used in sequencing, and thereby can directly be used in the subsequent computer sequencing.

Hereinafter, two examples of the method of DNA ligase mediated amplification and an disclosure of the method of DNA ligase mediated amplification as described in the present disclosure are briefly explained with reference to the drawings.

First Round Amplification

FIG. 1 is an exemplary embodiment of the first round amplification, wherein the first round amplification is a linear amplification. As FIG. 1 shows, the initial DNA template is a double-strand DNA template containing target region A. The primer pair in the first round amplification comprises an upstream primer and a downstream primer, wherein on the upstream primer from the 5′ end to the 3′ end successively are an adapter sequence and a hybrid sequence, wherein the hybrid sequence comprises random sequences N on two nonconsecutive base sites; and wherein on the downstream primer from the 5′ end to the 3′ end successively are a hybrid sequence and an adapter sequence, and the downstream primer contains a phosphate group at its 5′ end. An adapter sequence is selected so that it does hybridize with any site of the DNA template. A hybrid sequence of the upstream primer is selected so that it is completely complementary to the first nucleic acid sequence in target region A, except for on the base sites where two random sequences locate. A hybrid sequence of the downstream primer is selected so that it is completely complementary to the second nucleic acid sequence in target region A. Wherein the first nucleic acid sequence is downstream to the second nucleic acid sequence on the target DNA strand, and there are m nucleotides between the two regions.

N cycles of amplification steps were repeated (wherein N is an integer >1): 1) isolating the target DNA strand (e.g., antisense strand) from the double-strand DNA template through heat denaturation; 2) hybridizing the upstream primer and the downstream primer respectively to the first nucleic acid sequence and the second nucleic acid sequence within the target region A through annealing; 3) extending the upstream primer at the 3′ end to where immediately adjacent to the 5′ end of the downstream primer via DNA polymerase, to obtain extension product of the upstream primer; 4) ligating the extension product of the upstream primer to the downstream primer via heat resistant DNA ligase (e.g., Ampligase®); 5) repeating above steps 1)-4) N-1 times, where the difference is that in subsequent cycles, the double-strand DNA template of step 1) is the double stranded molecule obtained after the last cycle of amplification, which is formed by a half amplicon with adapter sequences at both ends and the initial target DNA strand. After N cycles of amplification, the linear amplification product contains N half amplicons.

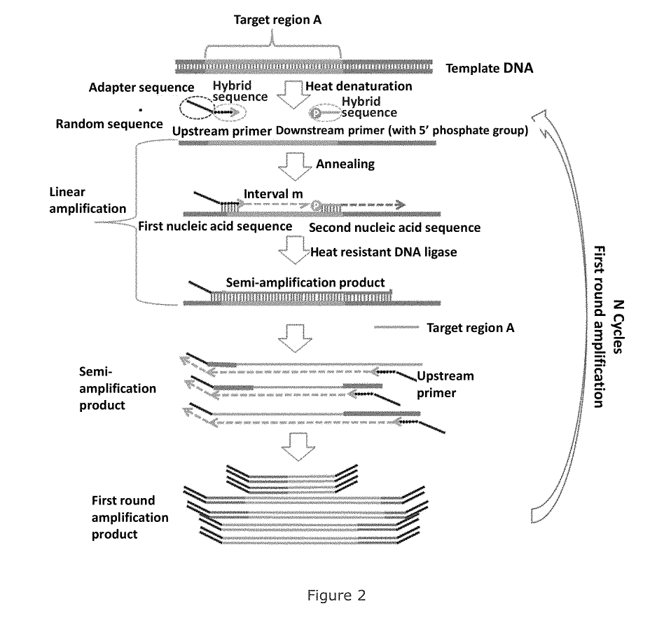

FIG. 2 is another exemplary embodiment of the first round amplification. As FIG. 2 shows, the initial DNA template is a double-strand DNA template containing target region A. The primer pair in the first round amplification comprises an upstream primer and a downstream primer, wherein on the upstream primer from the 5′ end to the 3′ end successively are an adapter sequence and a hybrid sequence, wherein the hybrid sequence comprises random sequences N on multiple consecutive base sites; wherein the downstream primer consists of or substantially consists of sequences reversely complementary to specific region of the target sequence, and the downstream primer contains a phosphate group at its 5′ end. An adapter sequence is selected so that it does hybridize with any site of the DNA template. A hybrid sequence of the downstream primer is selected so that it is completely or substantially completely complementary with the second nucleic acid sequence in target region A. The hybrid sequence of the upstream primer is completely or substantially completely complementary with the first nucleic acid sequence in target region A. Wherein the first nucleic acid sequence is downstream to the second nucleic acid sequence on the target DNA strand, and there are m nucleotides between the two regions.

N cycles of amplification steps were repeated (wherein N is an integer >1): 1) isolating the target DNA strand (e.g., antisense strand) from the double-strand DNA template through heat denaturation; 2) hybridizing the upstream primer and the downstream primer respectively to the first nucleic acid sequence and the second nucleic acid sequence within the target region A through annealing; 3) extending at 3′ end of the upstream primer and/or downstream primer via DNA polymerase, wherein the upstream primer is extended to where immediately adjacent to 5′ end of the downstream primer, to obtain extension product of the upstream primer; 4) ligating the extension product of the upstream primer to the downstream primer or extension product thereof via heat resistant DNA ligase (e.g.,)Ampligase®, to obtain semi-amplification product with adapter sequence at its 5′ end, which forms a double-strand molecule with the original target DNA strand; 5) a single-strand semi-amplification product is isolated from the double-strand molecule of step 4) through denaturing step, the upstream primer is hybridized to the single-strand semi-amplification product through annealing step, and the upstream primer is extended at 3′ end via a DNA polymerase, to obtain double-strand first round amplification product, with each strand comprising respectively an adapter sequence and a sequence complementary to the adapter sequence at the ends. Steps 1)-5) above are repeated N-1 times, where the difference is that in subsequent cycles, the double-strand DNA template of step 1) is the semi-amplification product or the first round amplification product obtained after the last round of amplification.

After multiple rounds of amplification, double-strand DNA molecules of different lengths which contain a part or entire of the target region are obtained.

Exponential Amplification

As FIG. 3 shows, after the first round amplification, exponential amplification is enabled by introducing exponential amplification primer pairs, wherein the exponential amplification primer pair comprises an exponential amplification upstream primer and an exponential amplification downstream primer. Wherein from the 5′ end to the 3′ end, the exponential amplification upstream primer successively contains sequences reversely complementary or identical to a part or entire of the sequencing upstream primer in the sequencing platform to be used (e.g. NGS sequencing), and sequence identical to or complementary to the adapter sequence (if any) of the first round amplification upstream and downstream primers. From the 5′ end to the 3′ end, the exponential amplification downstream primer successively contains sequences reversely complementary or identical to a part or entire of the sequencing downstream primer in the sequencing platform to be used (e.g. NGS sequencing), and sequences identical to or reversely complementary to the adapter sequence (if any) of the first round amplification upstream and downstream primer. The concept of the upstream and downstream primers herein is different from that of the upstream and downstream primers in linear amplification. The upstream and downstream primers of step ii) of the first round amplification are hybridized to the same target DNA strand, whereas the upstream and downstream primers in the exponential amplification can be respectively hybridized to the two strands of a double-strand DNA. In some embodiments, the sequences of the exponential amplification upstream and downstream primers are identical.

The first cycle of amplification in exponential amplification is performed by using the semi-amplification product or the first round amplification product obtained in the first round amplification as template, and every strand of DNA double stranded molecules formed during amplification can be used as template for the next cycle of amplification. Product obtained after multiple cycles of exponential amplification are sequences reversely complementary or identical to part or entire of the sequencing primer in the NGS sequencing platform at both ends, i.e., the formed amplification product is a DNA library that can directly be used in NGS sequencing.

Use of DNA Ligase Mediated Amplification Method in CNV Detection

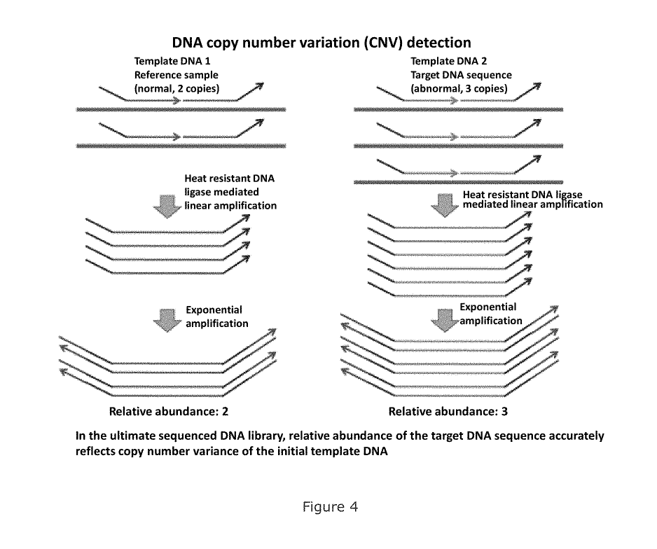

Shown in FIG. 4 is the use of the above DNA ligase mediated amplification method in CNV detection, which comprises steps of linear amplification and exponential amplification, wherein the target DNA sequence is hypothesized as template DNA 2, and the copy number of the DNA sequence in the biological sample where it derives from is expected to be determined. It is known that the copy number of the reference sample (template DNA 1) is 2. Two template DNAs are amplified in parallel DNA ligase mediated amplification experiments. As the figure shows, when the copy number of template DNA 2 is 3, after multiple cycles of linear amplification and then magnified through exponential amplification, the ratio between the abundance of the exponential amplification product of template DNA 2 and the abundance of the exponential amplification product of template DNA 1 is 3:2. From this abundance ratio, the copy number of template DNA 2 can be derived to be 3.

It should be understood that it is only an exemplary use of the amplification method of the present disclosure, which is not intended to limit the amplification method of the present disclosure to the above detection only. A person skilled in the art can flexibly choose the scope for applying the amplification method of the present disclosure based on practical need.

Another aspect of the present disclosure provides a kit used to amplify a target region at DNA level. In some embodiments, the kit comprises: the first round amplification primer pairs, wherein each of the primer pair comprises an upstream primer and a downstream primer, the upstream primer and downstream primer are hybridized to target DNA strand, wherein the upstream primer is hybridized to the first nucleic acid sequence of the target region, and the downstream primer is hybridized to the second nucleic acid sequence of the target region, wherein there are m nucleotides between the first nucleic acid sequence and the second nucleic acid sequence, and m is an integer ≧0, wherein the first nucleic acid sequence is downstream to the second nucleic acid sequence on the target DNA strand, and the downstream primer contains a phosphorylated 5′ end; and a ligating reagent, wherein the ligating reagent is used to ligate the upstream primer or extension product thereof to the downstream primer or extension product thereof to obtain a semi-amplification product.

In some embodiments, the ligating reagent comprises ligases and ligase reaction solutions. In some embodiments, the ligase is a heat resistant ligase. In some embodiments, the heat resistant ligase is Ampligase® DNA Ligase. In some embodiments, the heat resistant ligase is Taq DNA Ligase.

In some embodiments, the kit used to amplify a target region at DNA level further comprises: an extension reagent, the extension reagent is used to extend at 3′ end of the upstream primer and/or downstream primer using the antisense strand as template, to obtain extension products of the upstream primer and/or downstream primer. In some embodiments, the extension reagent comprises DNA polymerases, reaction reagents, and dNTP which consists of any one or more of dATP, dTTP, dGTP, and dCTP. In some embodiments, the upstream primer comprises adapter sequence at its 5′ end. In some embodiments, the upstream primer comprises adapter sequence at its 5′ end and the downstream primer comprises adapter sequence at its 3′ end. In some embodiments, the upstream primer comprises adapter sequence at its 5′ end and the downstream primer comprises no adapter sequence. In some embodiments, the first round amplification is a linear amplification, and the semi-amplification product is the first round amplification product. In some embodiments, the first round amplification is not a linear amplification, where the hybrid sequence comprised in the upstream primer comprises or is a random sequence, and the hybrid sequence comprised in the downstream primer is a hybrid sequence specifically recognizing the target region. When the upstream primer is hybridized to the semi-amplification product, the extension reagent can also be used to extend at 3′ end of the upstream primer using the semi-amplification product as template to obtain the first round amplification product.

In some embodiments, the kit used to amplify a target region at DNA level further comprises: an exponential amplification reagent, which is used to amplify the target region using the first round amplification product as template to obtain exponential amplification products.

In some embodiments, the exponential amplification reagent comprises DNA polymerases, reaction reagents and dNTP. In some embodiments, the exponential amplification reagent further comprises a universal primer for exponential amplifications, wherein the universal primer for exponential amplifications comprises a sequence identical to or reversely complementary to the 5′ end adapter sequence of the upstream primer in the first round amplification, and/or a sequence identical to or reversely complementary to the 3′ end adapter sequence of the downstream primer in the first round amplification.

In some embodiments, the kit used to amplify a target region at DNA level further comprises: a sequencing reagent, the sequencing reagent is used to sequence the semi-amplification product, the first round amplification product, or the exponential amplification product. In some embodiments, the exponential amplification reagent comprises a universal primer for exponential amplifications. In some embodiments, the exponential amplification reagent further comprises DNA polymerases, reaction reagents and dNTP.

### EXAMPLES

The present disclosure is further described in the following through some non-limited examples. It needs to be noted that these examples are only used to further illustrate the technical features of the present disclosure, which are not intended to be, nor can be interpreted as limited on the disclosure. These examples do not include elaboration of the traditional methods known to a person of skill in the art (extraction, purification, etc., of DNA in different types of samples).

### Example 1

### Use of the Ligate Mediated DNA Amplification Method in Detection for Chromosomal Copy Number Abnormity in Early Embryos

At present, in vitro fertilization has an success rate of 20%˜30%. Chromosomal aneuploidy (chromosomal number abnormity) is the major cause of in vitro fertilization failures, miscarriages, and abnormal pregnancies and live births in rare cases. The key to enhancing the success rate of in vitro fertilization is the selection of embryos with high quality. Data shows that about 60% of the three-day-old embryos have chromosomal abnormity, i.e., only about 40% embryos are normal. Therefore, before embryo implantation, chromosomal number of early embryos can be detected to screen for embryos with genetic abnormity, and thereby normal embryos are selected for implantation into the uterus, so that normal pregnancy and enhanced in vitro fertilization success rate can be expected.

The method of ligase mediated DNA amplification provided in the present disclosure can be used to accurately and speedily amplify a target region, and the copy number variation can be detected through subsequent sequencing of the target region.

The Initial DNA Template

Fertilized eggs were cultured in vitro. Single blastomeres in cleavage stage (e.g., within 24 hours of the in vitro culture), or multiple outer trophoblastic cells (1˜8 cells) in blastula stage (e.g., the third day of the in vitro culture) may be subject to detection for chromosomal copy number abnormity. The method of collecting blastomeres or outer trophoblastic cells may be any means known to a person skilled in the art, for example but not limited to the method recorded in Wang L, Cram DS et al. Validation of copy number variation sequencing for detecting chromosome imbalances in human preimplantation embryos. Biol Reprod, 2014, 91(2):37. Isolated blastomeres or trophoblastic cells were washed 3 times using PBS, and resuspended using 25 μl PBS solution, which was then directly applied to the first reaction solution system as the sample solution containing genome DNA. The initial sample solution contains about 3-24 pg total genome DNA.

Target Region

The detection is targeted at all 23 chromosomes, wherein one exemplary target region is the region of the LAMP2 gene on X chromosome (at base pairs 120442594-120442644 on X chromosome). The following primers are also correspondingly targeted at this exemplary target region.

### Reference Sample

A parallel experiment was conducted using a blood sample with known normal chromosomal copy number as reference . Where the first round amplification is linear amplification, the upstream primer and downstream primer both contain adapter sequences.

Linear Primer

The upstream primer (SEQ ID NO. 3) from the 5′ to the 3′ successively consists of an adapter sequence +a hybrid sequence (containing random sequences).

The adapter sequence is (5′ to 3′) SEQ ID NO. 1:
5′-CCTACACGACGCTCTTCCGATCT-3′.
The hybrid sequence is (5′ to 3′) SEQ ID NO. 2:
5′-CTTACCRGAGCCATTAACCAAATAC-3′.

The downstream primer (SEQ ID NO.6) consists of a hybrid sequence (containing no random sequences)+an adapter sequence from the 5′ to the 3′, and contains a 5′ phosphate group.

The hybrid sequence is (5′ to 3′) SEQ ID NO. 4:
5′-ATCTGAAGGAAGTGAACATCAGCAT-3′.
The adapter sequence is (5′ to 3′) SEQ ID NO. 5:
5′-GTGACTGGAGTTCAGACGTGTGC-3′.

Linear Amplification

The heat resistant ligase Ampligase® DNA Ligase (Epicentre, Wis., US) reaction system was adopted in linear amplification, wherein heat resistant DNA polymerases and reaction solution thereof in proper amount may be contained. Reaction conditions specific for ligase linear amplification were applied in the linear amplification, e.g., after initial 94° C. DNA denaturation for 2 minutes, steps of 94° C. denaturation for 30 seconds, 58° C. primer annealing, and ligation for 20 seconds, are repeated for 30 cycles.

Exponential Amplification

After the linear amplification, product of the linear amplification may be purified using a DNA purification kit and be used in the exponential amplification. Alternatively, exponential amplification may be performed by adding universal primer pairs for exponential amplification directly into the linear amplification system.

From 5′ to 3′, the exponential amplification upstream primer consists of an linker sequence necessary for sequencing+a sequence identical to the adapter sequence of the linear amplification upstream primer.

Linker sequence required for sequencing:
SEQ ID NO. 7:
5′-AATGATACGGCGACCACCGAGATCTACACACACTCTTTC-3′.
Sequence identical to the adapter sequence of the
linear amplification upstream primer SEQ ID NO. 8:
5′-CCTACACGACGCTCTTCCGATCT-3.
Exponential amplification upstream primer
SEQ ID NO. 9:
5′-AATGATACGGCGACCACCGAGATCTACACACACTCTTTCCCTAC
ACGACGCTCTTCCGATCT-3′.

From 5′ to 3′, the exponential amplification downstream primer consists of an linker sequence necessary for sequencing+a sequence reversely complementary to the adapter sequence of the linear amplification downstream primer.

Linker sequence required for sequencing SEQ ID
NO. 10:
5′-CAAGCAGAAGACGGCATACGAGATGATCGGAAGA-3′.
Sequence reversely complementary to the adapter
sequence of the linear amplification downstream
primer SEQ ID NO. 11:
5′-GCACACGTCTGAACTCCAGTCAC-3′.
Exponential amplification downstream primer SEQ
ID NO. 12:
5′-CAAGCAGAAGACGGCATACGAGATGATCGGAAGAGCACACGTCTGAA
CTCCAGTCAC-3′.

Reaction conditions applied in the exponential amplification are similar to the conditions applied in the classic polymerase chain reactions, e.g., using Taq polymerase, after initial 94° C. DNA denaturation for 2 minutes, steps of 94° C. denaturation for 30 seconds, 58° C. primer annealing for 20 seconds, and 65° C. extension for 30 seconds, are repeated for 30 cycles.

DNA Sequencing

Since exponential amplification product formed from the above exponential amplification steps contains sequences partly or completely complementary to the primer used for sequencing at both ends, such exponential amplification product may be regarded as a DNA library that can directly be used for sequencing. The DNA library was sequenced by high throughput DNA sequencing method. Prior to sequencing, the target DNA for sequencing may be enriched using an oligonucleotide probe.

The sequencing result was analyzed to determine the relative abundance of the reference sample and the sample to be tested respectively, and by comparing the relative abundance of the reference sample and the sample to be tested, the existence or not of the chromosomal copy number abnormity in the early embryo sample was determined.

### Example 2

### Designing and Using the Primers

In one embodiment of the present disclosure, the first round linear amplification uses an upstream primer A comprising a random sequence and an adapter sequence, and a downstream primer B specifically recognizing the target sequence. Below is an example for designing and evaluating the downstream primer B. Primer B was designed to specifically recognize chromosome related regions, which however does not recognize or seldom recognizes other locations in a genome. Within a human genome database, e.g., hg19 database, a designed primer B was blasted to obtain hg19 frequency which represents the number of perfect matches of primer B within an hg19 genome, meanwhile, a primer was blasted within the disease-corresponding target region to obtain MMS frequency which represents number of perfect matches within the chromosomal microdeletion and microduplication syndromes (MMS) target region. When the “hg19 frequency” is consistent with the “MMS frequency”, it means that primer B has specificity towards the MMS region. Conditioned upon that the primer has specificity, primers with greater number of perfect matches within the target MMS region are more desirable. In the present disclosure, one embodiment of amplifying the target region using aforesaid primer A and primer B is as follows: in the first round PCR, an upstream primer consisting of adapter sequence, endonuclease recognition sites, and random hexamers, from 5′ end to 3′ end, and a downstream primer specific for the target region (microdeletion and microduplication syndromes (MMS) related target region) were used, and by ligating the upstream primer and extension product thereof to the downstream primer to obtain the amplification product, which is a collection of fragments containing a specific region (primer B sequence), an adapter sequence and a sequence complementary to the adapter sequence at each end respectively (the first round amplification product). In the second round PCR, exponential amplification primer C was used to magnify all the signals to obtain an exponential amplification product comprising the entire adapter sequence and a part of the endonuclease recognition site sequence of the first round amplification upstream primer. After two rounds of PCR, the exponential amplification product can be digested using endonuclease, and the copy number of the target region in each sample can be detected via gel electrophoresis, or condition of the copy number of the target region in the samples can be detected through second generation sequencing.

Using cat eye syndrome (CES) as an example, which is related to copy number abnormity in certain regions of chromosome 22. According to aforesaid principles for design and evaluation, primers B which can be used in detecting cat eye syndrome are shown in table 1, and the locations of the corresponding sites on chromosome recognized by each primer B are shown in table 2.

TABLE 1
evaluation of primer B specific for cat eye syndrome

| | hg19 | MMS | Chromosomal | |
|---|---|---|---|---|
| Primer B | frequency | frequency | band | Location of chromosome |
| CTTCGATCACACG | 16 | 16 | 22q11.1 | chr22: 17565858-17591387 |
| (SEQ ID NO. 13) | | | | |
| ATCGCACACGCCC | 17 | 17 | 22q11.1 | chr22: 17565858-17591387 |
| (SEQ ID NO. 14) | | | | |

TABLE 2
location of sites on chromosome recognized by
primer B specific for cat eye syndrome

| Chromo- | Chromosome | Primer B | Positive or |
|---|---|---|---|
| some | location | sequence | antisense |
| chr22 | 17627601 | ATCGCACACGCCC | + |
| chr22 | 17628097 | ATCGCACACGCCC | + |
| chr22 | 17627787 | ATCGCACACGCCC | + |
| chr22 | 17628035 | ATCGCACACGCCC | + |
| chr22 | 17627725 | ATCGCACACGCCC | + |
| chr22 | 17627973 | ATCGCACACGCCC | + |
| chr22 | 17627663 | ATCGCACACGCCC | + |
| chr22 | 17627415 | ATCGCACACGCCC | + |
| chr22 | 17627911 | ATCGCACACGCCC | + |
| chr22 | 17628527 | ATCGCACACGCCC | + |
| chr22 | 17628403 | ATCGCACACGCCC | + |
| chr22 | 17628221 | ATCGCACACGCCC | + |
| chr22 | 17628465 | ATCGCACACGCCC | + |
| chr22 | 17628283 | ATCGCACACGCCC | + |
| chr22 | 17628159 | ATCGCACACGCCC | + |
| chr22 | 17627849 | ATCGCACACGCCC | + |
| chr22 | 17627539 | ATCGCACACGCCC | + |
| chr22 | 17628144 | CTTCGATCACACG | + |
| chr22 | 17627834 | CTTCGATCACACG | + |
| chr22 | 17628082 | CTTCGATCACACG | + |
| chr22 | 17627772 | CTTCGATCACACG | + |
| chr22 | 17628020 | CTTCGATCACACG | + |
| chr22 | 17627710 | CTTCGATCACACG | + |
| chr22 | 17628388 | CTTCGATCACACG | + |
| chr22 | 17628206 | CTTCGATCACACG | + |
| chr22 | 17628450 | CTTCGATCACACG | + |
| chr22 | 17627276 | CTTCGATCACACG | + |
| chr22 | 17627462 | CTTCGATCACACG | + |
| chr22 | 17627338 | CTTCGATCACACG | + |
| chr22 | 17628698 | CTTCGATCACACG | + |
| chr22 | 17627524 | CTTCGATCACACG | + |
| chr22 | 17627958 | CTTCGATCACACG | + |
| chr22 | 17628636 | CTTCGATCACACG | + |

Human peripheral blood DNA was prepared using DNeasy® tissue extraction kit (Qiagen). DNA sample was quantified using MBA 2000 spectrometer (PerkinElmer).

The following upstream and downstream primers were used in the first round PCR:

Upstream primer A (SEQ ID NO. 15):
5′-GTTCTACACGAGTCACTGCAGNNNNNNN-3′.
Downstream primer B (SEQ ID NO. 13):
5′-CTTCGATCACACG-3′.

In the first round amplification, heat resistant ligase Ampligase DNA Ligase (Epicentre) reaction system was used, wherein proper amount of heat resistant DNA polymerase and reaction solutions thereof can be included, and wherein reaction conditions specific for ligase mediated amplification were applied in the first round amplification, e.g., after initial 94° C. DNA denaturation for 2 minutes, steps of 94° C. denaturation for 30 seconds, 58° C. primer annealing, and ligation for 20 seconds, are repeated for 30 cycles.

Product of the first round PCR was purified using GENECLEAN® kit (Bio101). In the second round PCR, amplification was conducted using a specific primer C (SEQ ID NO.16): 5′-GTTCTACACGAGTCACTGC-3′, and using a quarter of the purified first round PCR product as template. To reduce background noises to the most extent, preparation and dilution of all the DNA samples must be carefully performed, and all reaction mixtures are prepared on a PCR worktable. The reaction solutions comprise ¼ purified the first round PCR product, 10 mM Tris-HCl, pH 8.3, 50 mM KCl, 2.5 mM MgCl₂, 200 μM dNTP of each type, 0.5 μM primer and 0.5 U Ampli-Taq® Gold DNA polymerase (PerkinElmer). The amplification reaction was performed in GeneAmp® 9600 automatic thermal cycler (PerkinElmer). Reaction conditions for the first round PCR were: 95° C. for 10 minutes, followed by 45 cycles of (95° C. for 1 minute, 55° C. for 1 minute, and 72° C. for 1 minute), and finally 72° C. extension for 5 minutes. Exponential amplification product was obtained after the completion of the second round PCR. Exponential amplification product was treated to prepare the second generation sequencing library and to be sequenced.

Reads in sequencing result containing primer B sequence will be recorded to characterize the copy number corresponding to the MMS region. During sequencing, normal double-copy regions that are not MMS regions were used as internal reference. The ratio between reads of MMS regions and reads of normal double-copy regions were evaluated using standard Z test, to determine the conditions of the copy number of MMS region (ratio>1 represents the increase of the copy number; ratio<1 represents the decrease of the copy number).

According to the method of design and evaluation as described in this embodiment, a series of downstream primers B specifically recognizing relevant regions of relevant diseases were selected regarding different CNV related diseases. The details are shown in table 3 below.

TABLE 3
specific downstream primers designed and selected for different CNV related
diseases

| CNV related diseases | hg19 position | Downstream primer B |
|---|---|---|
| DGS2 | chr10:21020310- | SEQ ID NO. 17: |
| | 21190925 | CCGTATCGGTTCC |
| DGS2 | chr10:21020310- | SEQ ID NO. 18: |
| | 21190925 | CCAAGACCCGTAC |
| DGS2 | chr10:21020310- | SEQ ID NO. 19: |
| | 21190925 | TAATAGGAACGCG |
| DGS2 | chr10:21020310- | SEQ ID NO. 20: |
| | 21190925 | ATGTAGTCGCCGT |
| DGS2 | chr10:21020310- | SEQ ID NO. 21: |
| | 21190925 | TTTCGTAGCGTGC |
| DGS2 | chr10:21020310- | SEQ ID NO. 22: |
| | 21190925 | ATACTGGCGAGTA |
| Hypoparathyroidism, nervous | chr10:8046416-8195974 | SEQ ID NO. 23: |
| deafness, and kidney diseases | | CGGCGCCGGACAA |
| Hypoparathyroidism, nervous | chr10:8046416-8195974 | SEQ ID NO. 24: |
| deafness, and kidney diseases | | CTCGTCGACCCAC |
| Hypoparathyroidism, nervous | chr10:8046416-8195974 | SEQ ID NO. 25: |
| deafness, and kidney diseases | | CGCGCGGTTAGCA |
| Hypoparathyroidism, nervous | chr10:8046416-8195974 | SEQ ID NO. 26: |
| deafness, and kidney diseases | | CGCGGTTAGCATG |
| 10q22-q23 microdeletion | chr10:81621602- | SEQ ID NO. 27: |
| syndrome | 81755912 | TAAGAGCGCGTTC |
| 10q22-q23 microdeletion | chr10:81621602- | SEQ ID NO. 28: |
| syndrome | 81755912 | ATCGTAGTGTACC |
| 10q22-q23 microdeletion | chr10:81621602- | SEQ ID NO. 29: |
| syndrome | 81755912 | CCGATGTGCGCAG |
| 10q22-q23 microdeletion | chr10:81621602- | SEQ ID NO. 30: |
| syndrome | 81755912 | CGTGCCCGCGTCA |
| Juvenile polyposis continuous | chr10:88626616- | SEQ ID NO. 31: |
| deletion syndrome | 88725624 | CGCCTGCGATTAT |
| Juvenile polyposis continuous | chr10:88626616- | SEQ ID NO. 32: |
| deletion syndrome | 88725624 | ATCTCGATACGAT |
| Juvenile polyposis continuous | chr10:88626616- | SEQ ID NO. 33: |
| deletion syndrome | 88725624 | CGAATCGGACGAG |
| Juvenile polyposis continuous | chr10:88626616- | SEQ ID NO. 34: |
| deletion syndrome | 88725624 | AATCGGACGAGAC |
| Juvenile polyposis continuous | chr10:89606978- | SEQ ID NO. 35: |
| deletion syndrome | 89808513 | ACTATGGTATCCG |
| Juvenile polyposis continuous | chr10:89606978- | SEQ ID NO. 36: |
| deletion syndrome | 89808513 | CGCTATACGGACT |
| SHFM3, FBW4 | chr10:102907119- | SEQ ID NO. 37: |
| | 102995110 | TCGCCGCCGGTTC |
| SHFM3, FBW4 | chr10:102907119- | SEQ ID NO. 38: |
| | 102995110 | GCCGCCGGTTCTA |
| SHFM3, FBW4 | chr10:103292101- | SEQ ID NO. 39: |
| | 103396606 | TAAATCACGGCGG |
| SHFM3, FBW4 | chr10:103292101- | SEQ ID NO. 40: |
| | 103396606 | GCGCGCTTAGCTA |
| SHFM3, FBW4 | chr11:103740036- | SEQ ID NO. 41: |
| | 103920683 | GCGCACTATCGAT |
| SHFM3, FBW4 | chr11:103740036- | SEQ ID NO. 42: |
| | 103920683 | CACGATACGGCCA |
| SHFM3, FBW4 | chr11:103740036- | SEQ ID NO. 43: |
| | 103920683 | AGCGCACTATCGA |
| SHFM3, FBW4 | chr11:103740036- | SEQ ID NO. 44: |
| | 103920683 | AGTCCAATTCGTG |
| POTOCKI-SHAFFER | chr11:43796310- | SEQ ID NO. 45: |
| syndrome | 43985276 | GTCGGCGACCCTT |
| POTOCKI-SHAFFER | chr11:43796310- | SEQ ID NO. 46: |
| syndrome | 43985276 | AGGACGACGCTAC |
| POTOCKI-SHAFFER | chr11:43796310- | SEQ ID NO. 47: |
| syndrome | 43985276 | TCGTCTGGTACGA |
| POTOCKI-SHAFFER | chr11:43796310- | SEQ ID NO. 48: |
| syndrome | 43985276 | TAAAGGGACGCGC |
| POTOCKI-SHAFFER | chr11:45850894- | SEQ ID NO. 49: |
| syndrome | 46013100 | CGTGGTTCGCGGC |
| POTOCKI-SHAFFER | chr11:45850894- | SEQ ID NO. 50: |
| syndrome | 46013100 | TCCTAACGCGCCG |
| POTOCKI-SHAFFER | chr11:45850894- | SEQ ID NO. 51: |
| syndrome | 46013100 | CAGTCGCTCGGTT |
| POTOCKI-SHAFFER | chr11:45850894- | SEQ ID NO. 52: |
| syndrome | 46013100 | CCGTGGTTCGCGG |
| PSS | chr11:46255040- | SEQ ID NO. 53: |
| | 46422796 | ATGCGCGCATGTC |
| PSS | chr11:46255040- | SEQ ID NO. 54: |
| | 46422796 | CGGGTCCACGACT |
| PSS | chr11:46255040- | SEQ ID NO. 55: |
| | 46422796 | CTCGCGAGTGTAC |
| PSS | chr11:46255040- | SEQ ID NO. 56: |
| | 46422796 | TCGCGAGTGTACA |
| PSS | chr11:44774296- | SEQ ID NO. 57: |
| | 44963698 | TTGCGTGACGCAG |
| PSS | chr11:44774296- | SEQ ID NO. 58: |
| | 44963698 | CAACGGCGTTGCT |
| PSS | chr11:44774296- | SEQ ID NO. 59: |
| | 44963698 | AACGGCGTTGCTA |
| PSS | chr11:44774296- | SEQ ID NO. 60: |
| | 44963698 | GCGTTGCTACACG |
| Type II aniridia (AN2) | chr11:31825658- | SEQ ID NO. 61: |
| | 31846431 | ACGAGTCGTCGAT |
| Type II aniridia (AN2) | chr11:31825658- | SEQ ID NO. 62: |
| | 31846431 | CGGCTAAACCCGC |
| Type II aniridia (AN2) | chr11:31825658- | SEQ ID NO. 63: |
| | 31846431 | GCGGCTCGTGCGT |
| WAGR syndrome | chr11:31825658- | SEQ ID NO. 64: |
| | 31846431 | ACGAGTCGTCGAT |
| WAGR syndrome | chr11:31825658- | SEQ ID NO. 65: |
| | 31846431 | CGGCTAAACCCGC |
| WAGR syndrome | chr11:31825658- | SEQ ID NO. 66: |
| | 31846431 | GCGGCTCGTGCGT |
| WAGR syndrome | chr11:32383408- | SEQ ID NO. 67: |
| | 32503936 | CACTAACTCGCGC |
| WAGR syndrome | chr11:32383408- | SEQ ID NO. 68: |
| | 32503936 | CACTAACTCGCGC |
| WAGR syndrome | chr11:32383408- | SEQ ID NO. 69: |
| | 32503936 | ACTAACTCGCGCC |
| WAGR syndrome | chr11:32383408- | SEQ ID NO. 70: |
| | 32503936 | ACTAACTCGCGCC |
| BECKWITH-WIEDEMAN | chr11:1830728-2019246 | SEQ ID NO. 71: |
| syndrome | | TCACGTACACCGC |
| BECKWITH-WIEDEMAN | chr11:1830728-2019246 | SEQ ID NO. 72: |
| syndrome | | ACGTACACCGCAG |
| BECKWITH-WIEDEMAN | chr11:1830728-2019246 | SEQ ID NO. 73: |
| syndrome | | CGGCTACGGAGAT |
| BECKWITH-WIEDEMAN | chr11:1830728-2019246 | SEQ ID NO. 74: |
| syndrome | | ACGCAAAGGCGAC |
| BECKWITH-WIEDEMAN | chr11:1830728-2019246 | SEQ ID NO. 75: |
| syndrome | | CTCTTAGCCGGTT |
| BECKWITH-WIEDEMAN | chr11:1830728-2019246 | SEQ ID NO. 76: |
| syndrome | | TAGCCGGTTAGGG |
| Microdeletion syndrome where | chr11:86592530- | SEQ ID NO. 77: |
| 11q14 contains exudative | 86745178 | CCCGCCGACGGTC |
| retinopathy | | |
| Microdeletion syndrome where | chr11:86592530- | SEQ ID NO. 78: |
| 11q14 contains exudative | 86745178 | CCGACGGTCTCGC |
| retinopathy | | |
| Microdeletion syndrome where | chr11:86592530- | SEQ ID NO. 79: |
| 11q14 contains exudative | 86745178 | CAGTGAAACGACG |
| retinopathy | | |
| Microdeletion syndrome where | chr11:86592530- | SEQ ID NO. 80: |
| 11q14 contains exudative | 86745178 | CAGCGGCGATCGG |
| retinopathy | | |
| 12q14 microdeletion syndrome | chr12:65499178- | SEQ ID NO. 81: |
| (BUSCHKE-OLLENDORFF | 65679006 | GAACGGAGCGCAT |
| syndrome) | | |
| 12q14 microdeletion syndrome | chr12:65499178- | SEQ ID NO. 82: |
| (BUSCHKE-OLLENDORFF | 65679006 | GCTTAGGCGCTTA |
| syndrome) | | |
| 12q14 microdeletion syndrome | chr12:65499178- | SEQ ID NO. 83: |
| (BUSCHKE-OLLENDORFF | 65679006 | GTCGAGCCTCGAG |
| syndrome) | | |
| 12q14 microdeletion syndrome | chr12:65499178- | SEQ ID NO. 84: |
| (BUSCHKE-OLLENDORFF | 65679006 | TCGAGCCTCGAGT |
| syndrome) | | |
| del(13)(q12.3q13.1) | chr13:32329404- | SEQ ID NO. 85: |
| microdeletion syndrome | 32445970 | CGGACGGCATTTC |
| del(13)(q12.3q13.2) | chr13:32329404- | SEQ ID NO. 86: |
| microdeletion syndrome | 32445970 | TATAACGTACGAA |
| del(13)(q12.3q13.3) | chr13:32329404- | SEQ ID NO. 87: |
| microdeletion syndrome | 32445970 | AACGTACGAAGTC |
| del(13)(q12.3q13.4) | chr13:32329404- | SEQ ID NO. 88: |
| microdeletion syndrome | 32445970 | GTACTATCGAACG |
| Retinoblastoma (RB1) | chr13:48862679- | SEQ ID NO. 89: |
| | 48999920 | TTAGTCGGCTTCG |
| Retinoblastoma (RB1) | chr13:48862679- | SEQ ID NO. 90: |
| | 48999920 | CACGCGATGCAAC |
| Retinoblastoma (RB1) | chr13:48862679- | SEQ ID NO. 91: |
| | 48999920 | TTTGCGGCGTTTC |
| Retinoblastoma (RB1) | chr13:48862679- | SEQ ID NO. 92: |
| | 48999920 | ACACGGTCGGTAA |
| 15q15.3 syndromic deafness and | chr15:43864136- | SEQ ID NO. 93: |
| infertility | 44008580 | CGTTCGAACGTGG |
| 15q15.4 syndromic deafness and | chr15:43864136- | SEQ ID NO. 94: |
| infertility | 44008580 | TCGAACGTGGCGA |
| 15q15.5 syndromic deafness and | chr15:43864136- | SEQ ID NO. 95: |
| infertility | 44008580 | ACCGCCCTCGCAT |
| 15q15.6 syndromic deafness and | chr15:43864136- | SEQ ID NO. 96: |
| infertility | 44008580 | CGAATACCGCCCT |
| 16q21-q22 microdeletion | chr16:66935252- | SEQ ID NO. 97: |
| syndrome | 67102915 | CGACCATGCGGCT |
| 16q21-q22 microdeletion | chr16:66935252- | SEQ ID NO. 98: |
| syndrome | 67102915 | ACGCAACGCCTCC |
| 16q21-q23 microdeletion | chr16:67102916- | SEQ ID NO. 99: |
| syndrome | 67218219 | CGAGCTTCGTGCG |
| MILLER-DIEKER | chr17:1164467-1352625 | SEQ ID NO. 100: |
| lissencephaly syndrome | | CGTGTAGCTCGAT |
| (MDLS) | | |
| MILLER-DIEKER | chr17:1061406-1124810 | SEQ ID NO. 101: |
| lissencephaly syndrome | | GAGTGATACGCGG |
| (MDLS) | | |
| MILLER-DIEKER | chr17:1061406-1124810 | SEQ ID NO. 102: |
| lissencephaly syndrome | | GTGATACGCGGAC |
| (MDLS) | | |
| MILLER-DIEKER | chr17:1061406-1124810 | SEQ ID NO. 103: |
| lissencephaly syndrome | | TGATACGCGGACA |
| (MDLS) | | |
| MILLER-DIEKER | chr17:1061406-1124810 | SEQ ID NO. 104: |
| lissencephaly syndrome | | GATACGCGGACAA |
| (MDLS) | | |
| Type I neurofibromatosis | chr17:29701179- | SEQ ID NO. 105: |
| | 29786697 | TCGCACAGACGTT |
| (chromosomal) deletion | chr1:2074487-2252409 | SEQ ID NO. 106: |
| syndrome | | CGGCGGCGATCTT |
| Cat eye syndrome (CES) | chr22:17503961- | SEQ ID NO. 107: |
| | 17654536 | ATCGCACACGCCC |
| Cat eye syndrome (CES) | chr22:17503961- | SEQ ID NO. 108: |
| | 17654536 | CTTCGATCACACG |
| Type I synpolydactyly (SPD1), | chr2:176880740- | SEQ ID NO. 109: |
| type D brachydactylia | 177056400 | GCCGAACCCGAGA |
| CR 3.2 Mb related speech delay | chr5:3003478-3186647 | SEQ ID NO. 110: |
| | | CACGAGTCGGTGC |
| Type I salt wasting syndrome | chr6:31873512- | SEQ ID NO. 111: |
| | 32052071 | TACGCCCAGGTAT |
| Type I salt wasting syndrome | chr6:31873512- | SEQ ID NO. 112: |
| | 32052071 | ATCGGGACCCGAT |
| Type I salt wasting syndrome | chr6:31873512- | SEQ ID NO. 113: |
| | 32052071 | ATCGAGGGTTACC |
| Type I salt wasting syndrome | chr6:31873512- | SEQ ID NO. 114: |
| | 32052071 | TCGCCGTCCACGA |
| 8p23.1 microdeletion and | chr8:11578760- | SEQ ID NO. 115: |
| microduplication syndrome | 11702671 | CTGTCGTGTGCGG |
| (MMS) | | |
| 9p24 sex reverse deletion | chr9:822247-1001152 | SEQ ID NO. 116: |
| syndrome | | ATGCGTGAAGTCG |
| 9q22.3 microdeletion syndrome | chr9:100785111- | SEQ ID NO. 117: |
| | 100984410 | ATGACTCCGACGT |
| 9q22.3 microdeletion syndrome | chr9:100043024- | SEQ ID NO. 118: |
| | 100206455 | CGGTTGAATAAGC |
| 9q22.3 microdeletion syndrome | chr9:100785111- | SEQ ID NO. 119: |
| | 100984410 | ACGCTGAAATCGG |
| DUCHENNE type | chrX:31147706- | SEQ ID NO. 120: |
| myodystrophia | 31333250 | GTACGCGGGCTTA |
| DUCHENNE type | chrX:31147706- | SEQ ID NO. 121: |
| myodystrophia | 31333250 | TATCGGACAAGGC |
| DUCHENNE type | chrX:31147706- | SEQ ID NO. 122: |
| myodystrophia | 31333250 | ATCATGAACGACG |
| DUCHENNE type | chrX:31147706- | SEQ ID NO. 123: |
| myodystrophia | 31333250 | ATTGTCAACGACC |
| Complicated glycerol kinase | chrX:30775010- | SEQ ID NO. 124: |
| deficiency | 30895166 | TAGGGTTACCGCC |
| Complicated glycerol kinase | chrX:30775010- | SEQ ID NO. 125: |
| deficiency | 30895166 | AGGTAGTCGCCTA |
| Complicated glycerol kinase | chrX:30775010- | SEQ ID NO. 126: |
| deficiency | 30895166 | TCGACAACGTTTC |
| Complicated glycerol kinase | chrX:30775010- | SEQ ID NO. 127: |
| deficiency | 30895166 | TCGAACACTGGTA |
| DSS repeats related adrenal | chrX:30436015- | SEQ ID NO. 128: |
| aplasia | 30553396 | GCTCGTTATAGAT |
| DSS repeats related adrenal | chrX:30436015- | SEQ ID NO. 129: |
| aplasia | 30553396 | TTCGAATTGACCG |
| DSS repeats related adrenal | chrX:30436015- | SEQ ID NO. 130: |
| aplasia | 30553396 | CGAATTGACCGTA |
| DSS repeats related adrenal | chrX:30436015- | SEQ ID NO. 131: |
| aplasia | 30553396 | TCGTGCTTCGGGC |
| X-linked ichthyosis | chrX:7182761-7340010 | SEQ ID NO. 132: |
| | | CGGAGTCTACGGG |
| X-linked ichthyosis | chrX:7182761-7340010 | SEQ ID NO. 133: |
| | | GCGGTTAACTAGT |
| X-linked ichthyosis | chrX:7182761-7340010 | SEQ ID NO. 134: |
| | | CGATTGCCAAACG |
| X-linked ichthyosis | chrX:7182761-7340010 | SEQ ID NO. 135: |
| | | GATGTCGATAACC |
| X-linked autism | chrX:5889414-5973357 | SEQ ID NO. 136: |
| | | AATCGTCTATGCG |
| X-linked autism | chrX:5889414-5973357 | SEQ ID NO. 137: |
| | | ATCGTCTATGCGC |
| X-linked autism | chrX:5889414-5973357 | SEQ ID NO. 138: |
| | | CGCCGTATGCAAC |
| X-linked autism | chrX:5889414-5973357 | SEQ ID NO. 139: |
| | | GCCTTAATCCGCT |

It should be understood that, the experimental operations described above are exemplary only. A person skilled in the art can perform any step above using any commercially available kit, wherein the reagents, reaction conditions, and reaction time etc., optionally used in these steps are different with each other but substantially have the same basic principles.

Although the means for performing some specific steps of the method disclosed in the present disclosure are described in details in the above examples, such description is only exemplary rather than limiting on the present disclosure. In fact, based on the examples of the present disclosure, a person of ordinary skill in the art can understand and perform other variations of the disclosed embodiments through studying the specification, the disclosure, the drawings, and the attached claims. In the claims, the term “comprise” does not preclude other elements and steps, and the terms “a,” and “an,” do not preclude plural forms. In the specification, “substantially” refers to the extent of more than 80%, more than 85%, more than 90%, more than 95%, more than 98%, or more than 99%.

1. A method of amplifying a target region at DNA level, said method comprising:
repeating N cycles of first round amplification, wherein N is an integral and 1≦N≦40, said first round amplification comprising:
i) denaturing DNA template to obtain an single target DNA strand;
ii) hybridizing first round amplification primer pairs to said target DNA strand to amplify said target region, each said first round amplification primer pair comprising an upstream primer and a downstream primer, said upstream primer hybridized to a first nucleic acid sequence of the target region, said downstream primer hybridized to a second nucleic acid sequence of the target region,
wherein said first nucleic acid sequence and said second nucleic acid sequence are on the same target DNA strand, and wherein there are m nucleotides between said first nucleic acid sequence and said second nucleic acid sequence, wherein m is an integral ≧0,
wherein said first nucleic acid sequence is downstream to said second nucleic acid sequence on the target DNA strand, and said downstream primer contains a phosphorylated 5′ end;
iii) optionally, extending at 3′ end of said upstream primer and/or said downstream primer using the target DNA strand as template;
iv) ligating the upstream primer or extension product thereof, to the downstream primer or extension product thereof to obtain an semi-amplification product.

2-5. (canceled)

6. The method according to claim 1, wherein said upstream primer comprises adapter sequence at its 5′ end.

7. The method according to claim 1, wherein said upstream primer and said downstream primer both comprise adapter sequences, wherein said upstream primer comprises adapter sequence at its 5′ end and said downstream primer comprises adapter sequence at its 3′ end.

8. The method according to claim 1, wherein the ligating step is performed via a heat resistant ligase.

9. The method according to claim 1, wherein further said downstream primer comprises hybridization sequence that specifically recognizing said target region, said upstream primer comprises random sequence.

10. The method according to claim 9, wherein said first round amplification further comprises:
v) hybridizing said upstream primer to said semi-amplification product, and extending at 3′ end of the upstream primer using semi-amplification product as template to obtain a first round amplification product.

11. The method according to claim 1, further comprising:
sequencing said semi-amplification product to determine sequence of said target region.

12. The method according to claim 10, further comprising:
sequencing said first round amplification product to determine sequence of said target region.

13. The method according to claim 1, further comprising:
using amplification product of the first round amplification product as template, amplifying said target region through polymerase chain reaction (PCR), to generate an exponential amplification product.

14. The method according to claim 13, further comprising sequencing said exponential amplification product to determine sequence of said target region.

15-17. (canceled)

18. A kit used to amplify a target region at DNA level, wherein said kit comprises:
a first round amplification primer pair,
wherein said primer pair comprises an upstream primer and a downstream primer, said upstream primer and downstream primer hybridized to target DNA strand, wherein said upstream primer is hybridized to a first nucleic acid sequence of said target region, and said downstream primer is hybridized to a second nucleic acid sequence of said target region,
wherein there are m nucleotides between said first nucleic acid sequence and said second nucleic acid sequence, wherein m is an integral ≧0,
wherein said first nucleic acid sequence is downstream to said second nucleic acid sequence on the target DNA strand, and said downstream primer contains a phosphorylated 5′ end; and
a ligating reagent,
wherein said ligating reagent is used to ligate said upstream primer or extension product thereof to said downstream primer or extension product thereof to obtain an semi-amplification product.

19. The kit according to claim 18, wherein said ligating reagent comprises ligases and ligase reaction solutions, wherein said ligase is a heat resistant ligase.

20-22. (canceled)

23. The kit according to claim 18, further comprising:
an extension reagent, wherein said extension reagent comprises DNA polymerases, reaction solutions, and any one or more of dATP, dTTP, dGTP, and dCTP.

24. The kit according to claim 18, wherein said upstream primer comprises adapter sequence at its 5′ end, and/or said downstream primer comprises adapter sequence at its 3′ end.

25-26. (canceled)

27. The kit according to claim 24, further comprising:
a universal primer for exponential amplifications, wherein said exponential amplification reagent comprises a universal primer for exponential amplifications, wherein said universal primer contains a sequence identical to or reversely complementary to the 5′ end adapter sequence of said upstream primer, and/or a sequence identical to or reversely complementary to the 3′ end adapter sequence of said downstream primer.

28. The kit according to claim 18, further comprising:
a sequencing reagent.
